SEQUENCE LISTING (5 sequences)

SEQ ID NO: 1 - protein, 113 aa, Artificial Sequence
  Murine light chain sequence
DIQLTQSPSSLAVSAGENVTMSCKSSQSVLYSANHKNYLAWYQQKPGQSPKLLIYWASTRESGVPDRFTGSGSGTDFTLTISRVQVEDLAIYYCHQYLSSWTFGGGTKLEIKR

SEQ ID NO: 2 - protein, 114 aa, Artificial Sequence
  Humanised light chain sequence
DIQLTQSPSSLAVSAGVNDRMSCKSSQSVLYSANHKNYLAWYQQKPGQSKAKLLIYWASTRESGVPDSFTSSGSGTDFTLFISRSLVPDLIITYCHQYLSSWTFGGGTKLEIKR

SEQ ID NO: 3 - protein, 116 aa, Artificial Sequence
  Murine heavy chain sequence
QVQLQESGAELSKPGASVKMSCKASGYTFTSYWLHWIKQRPGQGLEWIGYINPRNDYTEYNQNFKDKATLTADKSSSTAYMQLSSLTSEDSAVYYCARRDITTFYWGQGTTLTVSS

SEQ ID NO: 4 - protein, 116 aa, Artificial Sequence
  First humanised heavy chain sequence
QVQLQQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTTVTVSS

SEQ ID NO: 5 - protein, 116 aa, Artificial Sequence [sequence omitted]

(19) United States
(12) Patent Application Publication
(10) Pub. No.: US 2021/0322583 A1
(43) Pub. Date: Oct. 21, 2021

(54) RADIO-PHARMACEUTICAL COMPLEXES

(71) Applicant: Bayer AS, Oslo (NO)

(72) Inventor: Alan CUTHBERTSON, Oslo (NO)

(73) Assignee: Bayer AS, Oslo (NO)

(21) Appl. No.: 17/150,811

(22) Filed: Jan. 15, 2021

Related U.S. Application Data
(63) Continuation of application No. 15/537,127, filed on Jun. 16, 2017, now abandoned.

(30) Foreign Application Priority Data
Dec. 17, 2014 (GB) 1422512.2

(51) Int. Cl. A61K 51/10

(52) U.S. Cl. CPC A61K 51/1093; A61K 51/1021; A61K 51/1072; A61K 51/1051; A61K 51/1069; A61K 51/103

(57) ABSTRACT

The invention provides a method for the formation of a tissue-targeting thorium complex, said method comprising: a) forming an octadentate chelator comprising four hydroxypyridinone (HOPO) moieties, substituted in the N-position with a C₁-C₃alkyl group, and a coupling moiety terminating in a carboxylic acid group; b) coupling said octadentate chelator to at least one tissue-targeting peptide or protein comprising at least one amine moiety by means of at least one amide-coupling reagent whereby to generate a tissue-targeting chelator; and c) contacting said tissue-targeting chelator with an aqueous solution comprising an ion of at least one alpha-emitting thorium isotope. A method of treatment of a neoplastic or hyperplastic disease comprising administration of such a tissue-targeting thorium complex, as well as the complex and corresponding pharmaceutical formulations are also provided.

### CROSS-REFERENCE TO RELATED APPLICATIONS

This application is a continuation application of U.S. patent application Ser. No. 15/537,127, which adopts the international filing date of Dec. 15, 2015, which is the National Phase application under 35 U.S.C. § 371 of International Application No. PCT/EP2015/079773, filed Dec. 15, 2015, which claims priority benefit to GB Application No. 1422512.2, filed Dec. 17, 2014.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 777052031401SEQLIST.TXT; date recorded: Jan. 14, 2021; size: 6 KB).

### FIELD OF THE INVENTION

The present invention relates to methods for the formation of complexes of thorium isotopes and particularly complexes of thorium-227 with certain octadentate ligands conjugated to tissue targeting moieties. The invention also relates to the complexes, and to the treatment of diseases, particularly neoplastic diseases, involving the administration of such complexes.

### BACKGROUND TO THE INVENTION

Specific cell killing can be essential for the successful treatment of a variety of diseases in mammalian subjects. Typical examples of this are in the treatment of malignant diseases such as sarcomas and carcinomas. However the selective elimination of certain cell types can also play a key role in the treatment of other diseases, especially hyperplastic and neoplastic diseases.

The most common methods of selective treatment are currently surgery, chemotherapy and external beam irradiation. Targeted radionuclide therapy is, however, a promising and developing area with the potential to deliver highly cytotoxic radiation specifically to cell types associated with disease. The most common forms of radiopharmaceuticals currently authorised for use in humans employ beta-emitting and/or gamma-emitting radionuclides. There has, however, been some interest in the use of alpha-emitting radionuclides in therapy because of their potential for more specific cell killing.

The radiation range of typical alpha emitters in physiological surroundings is generally less than 100 micrometers, the equivalent of only a few cell diameters. This makes these sources well suited for the treatment of tumours, including micrometastases, because they have the range to reach neighbouring cells within a tumour but if they are well targeted then little of the radiated energy will pass beyond the target cells. Thus, not every cell need be targeted but damage to surrounding healthy tissue may be minimised (see Feinendegen et al., Radiat Res 148:195-201 (1997)). In contrast, a beta particle has a range of 1 mm or more in water (see Wilbur, Antibody Immunocon Radiopharm 4: 85-96 (1991)).

The energy of alpha-particle radiation is high in comparison with that carried by beta particles, gamma rays and X-rays, typically being 5-8 MeV, or 5 to 10 times that of a beta particle and 20 or more times the energy of a gamma ray. Thus, this deposition of a large amount of energy over a very short distance gives α-radiation an exceptionally high linear energy transfer (LET), high relative biological efficacy (RBE) and low oxygen enhancement ratio (OER) compared to gamma and beta radiation (see Hall, “Radiobiology for the radiologist”, Fifth edition, Lippincott Williams & Wilkins, Philadelphia Pa., USA, 2000). This explains the exceptional cytotoxicity of alpha emitting radionuclides and also imposes stringent demands on the biological targeting of such isotopes and upon the level of control and study of alpha emitting radionuclide distribution which is necessary in order to avoid unacceptable side effects.

Table 1 below shows the physical decay properties of the alpha emitters so far broadly proposed in the literature as possibly having therapeutic efficacy.

TABLE 1

| Candidate nuclide | T_{1/2}* | | Clinically tested for |
|---|---|---|---|
| ²²⁵Ac | 10.0 | days | leukaemia |
| ²¹¹At | 7.2 | hours | glioblastoma |
| ²¹³Bi | 46 | minutes | leukaemia |
| ²²³Ra | 11.4 | days | skeletal metastases |
| ²²⁴Ra | 3.66 | days | ankylosing spondylitis |

*Half life

So far, with regards to the application in radioimmunotherapy the main attention has been focused on ²¹¹At, ²¹³Bi and ²²⁵Ac and these three nuclides have been explored in clinical immunotherapy trials.

Several of the radionuclides which have been proposed are short-lived, i.e. have half-lives of less than 12 hours. Such a short half-life makes it difficult to produce and distribute radiopharmaceuticals based upon these radionuclides in a commercial manner. Administration of a short-lived nuclide also increases the proportion of the radiation dose which will be emitted in the body before the target site is reached.

The recoil energy from alpha-emission will in many cases cause the release of daughter nuclides from the position of decay of the parent. This recoil energy is sufficient to break many daughter nuclei out from the chemical environment which may have held the parent, e.g. where the parent was complexed by a ligand such as a chelating agent.

This will occur even where the daughter is chemically compatible with, i.e. complexable by, the same ligand. Equally, where the daughter nuclide is a gas, particularly a noble gas such as radon, or is chemically incompatible with the ligand, this release effect will be even greater. When daughter nuclides have half-lives of more than a few seconds, they can diffuse away into the blood system, unrestrained by the complexant which held the parent. These free radioactive daughters can then cause undesired systemic toxicity.

The use of Thorium-227 (T_{1/2}=18.7 days) under conditions where control of the ²²³Ra daughter isotope is maintained was proposed a few years ago (see WO 01/60417 and WO 02/05859). This was in situations where a carrier system is used which allows the daughter nuclides to be retained by a closed environment. In one case, the radionuclide is disposed within a liposome and the substantial size of the liposome (as compared to recoil distance) helps retain daughter nuclides within the liposome. In the second case, bone-seeking complexes of the radionuclide are used which incorporate into the bone matrix and therefore restrict release of the daughter nuclides. These are potentially highly advantageous methods, but the administration of liposomes is not desirable in some circumstances and there are many diseases of soft tissue in which the radionuclides cannot be surrounded by a mineralised matrix so as to retain the daughter isotopes.

More recently, it was established that the toxicity of the ²²³Ra daughter nuclei released upon decay of ²²⁷Th could be tolerated in the mammalian body to a much greater extent than would be predicted from prior tests on comparable nuclei. In the absence of the specific means of retaining the radium daughters of thorium-227 discussed above, the publicly available information regarding radium toxicity made it clear that it was not possible to use thorium-227 as a therapeutic agent since the dosages required to achieve a therapeutic effect from thorium-227 decay would result in a highly toxic and possibly lethal dosage of radiation from the decay of the radium daughters, i.e. there is no therapeutic window.

WO 04/091668 describes the unexpected finding that a therapeutic treatment window does exist in which a therapeutically effective amount of a targeted thorium-227 radionuclide can be administered to a subject (typically a mammal) without generating an amount of radium-223 sufficient to cause unacceptable myelotoxicity. This can therefore be used for treatment and prophylaxis of all types of diseases at both bony and soft-tissue sites.

In view of the above developments, it is now possible to employ alpha-emitting thorium-227 nuclei in endoradionuclide therapy without lethal myelotoxicity resulting from the generated ²²³Ra. Nonetheless, the therapeutic window remains relatively narrow and it is in all cases desirable to administer no more alpha-emitting radioisotope to a subject than absolutely necessary. Useful exploitation of this new therapeutic window would therefore be greatly enhanced if the alpha-emitting thorium-227 nuclei could be complexed and targeted with a high degree of reliability.

Because radionuclides are constantly decaying, the time spent handling the material between isolation and administration to the subject is of great importance. It would also be of considerable value if the alpha-emitting thorium nuclei could be complexed, targeted and/or administered in a form which was quick and convenient to prepare, preferably requiring few steps, short incubation periods and/or temperatures not irreversibly affecting the properties of the targeting entity. Furthermore, processes which can be conducted in solvents that do not need removal before administration (essentially in aqueous solution) have the considerable advantage of avoiding a solvent evaporation or dialysis step.

It would also be considered of significant value if a thorium labelled drug product formulation could be developed which demonstrated significantly enhanced stability.

This is critical to ensure that robust product quality standards are adhered to while at the same time enabling a logistical path to delivering patient doses. Thus formulations with minimal radiolysis over a period of 1-4 days are preferred.

Octadentate chelating agents containing hydroxypyridinone groups have previously been shown to be suitable for coordinating the alpha emitter thorium-277, for subsequent attachment to a targeting moiety (WO2011098611). Octadentate chelators were described, containing four 3,2-hydroxypyridinone groups joined by linker groups to an amine-based scaffold, having a separate reactive group used for conjugation to a targeting molecule. Preferred structures of the previous invention contained 3,2-hydroxypyridinone groups and employed the isothiocyanate moiety as the preferred coupling chemistry to the antibody component as shown in compound ALG-DD-NCS. The isothiocyanate is widely used to attach a label to proteins via amine groups. The isothiocyanate group reacts with amino terminal and primary amines in proteins and has been used for the labelling of many proteins including antibodies. Although the thiourea bond formed in these conjugates is reasonably stable, it has been reported that antibody conjugates prepared from fluorescent isothiocyanates deteriorate over time. [Banks P R, Paquette D M., Bioconjug Chem (1995) 6:447-458]. The thiourea formed by the reaction of fluorescein isothiocyanate with amines is also susceptible to conversion to a guanidine under basic conditions [Dubey I, Pratviel G, Meunier B Journal: Bioconjug Chem (1998) 9:627-632]. Due to the long decay half-life of thorium-227 (18.7 days) coupled to the long biological half-life of a monoclonal antibody it is desirable to use more stable linking moieties so as to generate conjugates which are more chemically stable both in vivo and to storage.

The most relevant previous work on conjugation of hydroxypyridinone ligands was published in WO2013/167754 and discloses ligands possessing a water solubilising moiety comprising a hydroxyalkyl functionality. Due to the reactivity of the hydroxyl groups of this chelate class activation as an activated ester is not possible as multiple competing reactions ensue leading to a complex mixture of products through esterification reactions. The ligands of WO2013/167754 must therefore be coupled to the tissue-targeting protein via alternative chemistries such as the isothiocyanate giving a less stable thiourea conjugate as described above. In addition WO2013167755 and WO2013167756 discloses the hydroxyalkyl/isothiocyanate conjugates applied to CD33 and CD22 targeted antibodies respectively.

The present inventors have now established that by forming a tissue targeting complex by coupling specific chelators to appropriate targeting moieties, followed by addition of an alpha-emitting thorium ion, a complex may be generated rapidly, under mild conditions and by means of a linking moiety that remains more stable to storage and administration of the complex.

### SUMMARY OF THE INVENTION

In a first aspect, the present invention therefore provides a method for the formation of a tissue-targeting thorium complex, said method comprising:
a) forming an octadentate chelator comprising four hydroxypyridinone (HOPO) moieties, substituted in the N-position with a C₁-C₃ alkyl group, and coupling moiety terminating in a carboxylic acid group (or protected equivalent thereof);
b) coupling said octadentate chelator to at least one tissue-targeting peptide or protein comprising at least one amine moiety by means of at least one amide-coupling reagent whereby to generate a tissue-targeting chelator; and
c) contacting said tissue-targeting chelator with an aqueous solution comprising an ion of at least one alpha-emitting thorium isotope.

In such complexes (and preferably in all aspects of the current invention) the thorium ion will generally be complexed by the octadentate hydroxypyridinone-containing ligand, which in turn will be attached to the tissue targeting moiety via an amide bond.

Typically, the method will be a method for the synthesis of 3,2-hydroxypyridinone-based octadentate chelates comprising a reactive carbon/late function which can be activated in the form of an active ester (such as an N-hydroxysuccinimide ester (NHS ester)) either via in situ activation or by synthesis and isolation of the active ester itself.

The resulting NHS ester can be used in a simple conjugation step to produce a wide range of chelate modified protein formats. In addition, highly stable antibody conjugates are readily labelled with thorium-227. This may be at or close to ambient temperature, typically in high radiochemical yields and purity.

The method of the invention will preferably be carried out in aqueous solution and in one embodiment may be carried out in the absence or substantial absence (less than 1% by volume) of any organic solvent.

Preferred targeting moieties include polyclonal and particularly monoclonal antibodies and fragments thereof. Specific binding fragments such as Fab, Fab′, F(ab′)₂ and single-chain specific binding antibodies are typical fragments.

The tissue targeting complexes of the present invention may be formulated into medicaments suitable for administration to a human or non-human animal subject.

In a second aspect the invention therefore provides methods for the generation of a pharmaceutical formulation comprising forming a tissue-targeting complex as described herein followed by addition of at least one pharmaceutical carrier and/or excipient. Suitable carriers and excipients include buffers, chelating agents, stabilising agents and other suitable components known in the art and described in any aspect herein.

In a further aspect, the invention additionally provides a tissue-targeting thorium complex. Such a complex will have the features described herein throughout, particularly the preferred features described herein. The complex may be formed or formable by any of the methods described herein. Such methods may thus yield at least one tissue-targeting thorium complex as described in any aspect or embodiment herein.

In a still further aspect, the present invention provides a pharmaceutical formulation comprising any of the complexes described herein. The formulation may be formed or formable by any of the methods described herein and may contain at least one buffer, stabiliser and/or excipient. The choice of buffer and stabiliser may be such that together they help to protect the tissue-targeting complex from radiolysis. In one embodiment, radiolysis of the complex in the formulation is minimal even after several days post manufacture of the formulation. This is an important advantage because it solves potential issues associated with product quality and the logistics of drug supply which are key to enablement and practical application of this technology.

This invention has shown utility in the preparation of a multitude of thorium-labelled antibody conjugates for the targeting of sites of biological interest, such as tumour associated receptors.

### DETAILED DESCRIPTION OF THE INVENTION

In the context of the present invention, “tissue targeting” is used herein to indicate that the substance in question (particularly when in the form of a tissue-targeting complex as described herein), serves to localise itself (and particularly to localise any conjugated thorium complex) preferentially to at least one tissue site at which its presence (e.g. to deliver a radioactive decay) is desired. Thus a tissue targeting group or moiety serves to provide greater localisation to at least one desired site in the body of a subject following administration to that subject in comparison with the concentration of an equivalent complex not having the targeting moiety. The targeting moiety in the present case will be preferably selected to bind specifically to cell-surface receptors associated with cancer cells or other receptors associated with the tumour microenvironment.

There are a number of targets which are known to be associated with hyperplastic and neoplastic disease. These include certain receptors, cell surface proteins, transmembrane proteins and proteins/peptides found in the extracellular matrix in the vicinity of diseased cells. Examples of cell-surface receptors and antigens which may be associated with neoplastic disease include CD22, CD33, FGFR2 (CD332), PSMA, HER2, Mesothelin etc. In one embodiment, the tissue-targeting moiety (e.g. peptide or protein) has specificity for at least one antigen or receptor selected from CD22, CD33, FGFR2 (CD332), PSMA, HER2 and Mesothelin.

CD22, or cluster of differentiation-22, is a molecule belonging to the SIGLEC family of lectins (SIGLEC=Sialic acid-binding immunoglobulin-type lectins).

CD33 or Siglec-3 is a transmembrane receptor expressed on cells of myeloid lineage.

FGFR2 is a receptor for fibroblast growth factor. It is a protein that in humans is encoded by the FGFR2 gene residing on chromosome 10.

HER2 is a member of the human epidermal growth factor receptor (HER/EGFR/ERBB) family.

Prostate-specific membrane antigen (PSMA) is an enzyme that in humans is encoded by the FOLH1 (folate hydrolase 1) gene.

Mesothelin, also known as MSLN, is a protein that in humans is encoded by the MSLN gene.

A particularly preferred tissue-targeting binder in the present case will be selected to bind specifically to CD22 receptor. This may be reflected, for example by having 50 or more times greater binding affinity for cells expressing CD22 than for non-CD22 expressing cells (e.g. at least 100 time greater, preferably at least 300 times greater). It is believed that CD22 is expressed and/or over-expressed in cells having certain disease states (as indicated herein) and thus the CD22 specific binder may serve to target the complex to such disease-affected cells. Similarly a tissue targeting moiety may bind to cell-surface markers (e.g. CD22 receptors) present on cells in the vicinity of disease affected cells. CD22 cell-surface markers may be more heavily expressed on diseased cell surfaces than on healthy cell surfaces or more heavily expressed on cell surfaces during periods of growth or replication than during dormant phases. In one embodiment, a CD22 specific tissue-targeting binder may be used in combination with another binder for a disease-specific cell-surface marker, thus giving a dual-binding complex. Tissue-targeting binders for CD-22 will typically be peptides or proteins, as discussed herein.

The various aspects of the invention as described herein relate to treatment of disease, particularly for the selective targeting of diseased tissue, as well as relating to complexes, conjugates, medicaments, formulation, kits etc. useful in such methods. In all aspects, the diseased tissue may reside at a single site in the body (for example in the case of a localised solid tumour) or may reside at a plurality of sites (for example where several joints are affected in arthritis or in the case of a distributed or metastasised cancerous disease).

The diseased tissue to be targeted may be at a soft tissue site, at a calcified tissue site or a plurality of sites which may all be in soft tissue, all in calcified tissue or may include at least one soft tissue site and/or at least one calcified tissue site. In one embodiment, at least one soft tissue site is targeted. The sites of targeting and the sites of origin of the disease may be the same, but alternatively may be different (such as where metastatic sites are specifically targeted). Where more than one site is involved this may include the site of origin or may be a plurality of secondary sites.

The term “soft tissue” is used herein to indicate tissues which do not have a “hard” mineralised matrix. In particular, soft tissues as used herein may be any tissues that are not skeletal tissues. Correspondingly, “soft tissue disease” as used herein indicates a disease occurring in a “soft tissue” as used herein. The invention is particularly suitable for the treatment of cancers and “soft tissue disease” thus encompasses carcinomas, sarcomas, myelomas, leukemias, lymphomas and mixed type cancers occurring in any “soft” (i.e. non-mineralised) tissue, as well as other non-cancerous diseases of such tissue. Cancerous “soft tissue disease” includes solid tumours occurring in soft tissues as well as metastatic and micro-metastatic tumours. Indeed, the soft tissue disease may comprise a primary solid tumour of soft tissue and at least one metastatic tumour of soft tissue in the same patient. Alternatively, the “soft tissue disease” may consist of only a primary tumour or only metastases with the primary tumour being a skeletal disease. Particularly suitable for treatment and/or targeting in all appropriate aspects of the invention are hematological neoplasms and especially neoplastic diseases of lymphoid cells, such as lymphomas and lymphoid leukemias, including Non-Hodgkin's Lymphoma, B-cell neoplasms of B-cell lymphomas. Similarly, any neoplastic diseases of bone marrow, spine (especially spinal cord) lymph nodes and/or blood cells are suitable for treatment and/or targeting in all appropriate aspects of the invention.

Some examples of B-cell neoplasms that are suitable for treatment and/or targeting in appropriate aspects of the present invention include:

Chronic lymphocytic leukemia/Small lymphocytic lymphoma, B-cell prolymphocytic leukemia, Lymphoplasmacytic lymphoma (such as Waldenström macroglobulinemia), Splenic marginal zone lymphoma, Plasma cell neoplasms (e.g. Plasma cell myeloma, Plasmacytoma, Monoclonal immunoglobulin deposition diseases, Heavy chain diseases), Extranodal marginal zone B cell lymphoma (MALT lymphoma), Nodal marginal zone B cell lymphoma (NMZL), Follicular lymphoma, Mantle cell lymphoma, Diffuse large B cell lymphoma, Mediastinal (thymic) large B cell lymphoma, Intravascular large B cell lymphoma, Primary effusion lymphoma and Burkitt lymphoma/leukemia.

Some examples of neoplasms suitable for treatment using a FGFR2 targeting agent of the present invention include those where mutational events are associated with tumour formation and progression including breast, endometrial and gastric cancers.

Some examples of myeloid derived neoplasms suitable for treatment using a CD33 targeted agent of the present invention includes Acute Myeloid Leukemia (AML).

Some further examples of neoplasms suitable for treatment using a prostate specific membrane antigen (PSMA) targeted agent of the present invention includes prostate and brain cancers.

Some further examples of neoplasms suitable for treatment using a Human Epidermal Growth Factor Receptor-2 (HER-2) targeted agent of the present invention includes breast cancers.

Some further examples of neoplasms suitable for treatment using a mesothelin targeted agent of the present invention include malignancies such as mesothelioma, ovarian, lung and pancreatic cancer,

It is a key contribution to the success of this invention that the antibody conjugates are stable for acceptable periods of time on storage. Hence the stability of both the non-radioactive antibody conjugate and the final thorium-labelled drug product must meet the stringent criteria demanded for manufacture and distribution of radiopharmaceutical products. It was a surprising finding that the formulation described herein comprising a tissue-targeting demonstrates outstanding stability on storage. This applies even at the elevated temperatures typically used for accelerated stability studies.

In one embodiment applicable to all compatible aspects of the invention, the tissue-targeting complex may be dissolved in a suitable buffer. In particular, it has been found that the use of a citrate buffer provides a surprisingly stable formulation. This is preferably citrate buffer in the range 1-100 mM (pH 4-7), particularly in the range 10 to 50 mM, but most preferably 20-40 mM citrate buffer.

In a further embodiment applicable to all compatible aspects of the invention, the tissue-targeting complex may be dissolved in a suitable buffer containing p-aminobutyric acid (PABA). A preferred combination is citrate buffer (preferably at the concentrations described herein) in combination with PABA. Preferred concentrations for PABA for use in any aspect of the present invention, including in combination with other agents is around 0.005 to 5 mg/ml, preferably 0.01 to 1 mg/ml and more preferably 0.01 to 1 mg/ml. Concentrations of 0.1 to 0.5 mg/ml are most preferred.

In a further embodiment applicable to all compatible aspects of the invention, the tissue-targeting complex may be dissolved in a suitable buffer containing ethylenediaminetetraacetic acid (EDTA). A preferred combination is the use of EDTA with citrate buffer. A particularly preferred combination is the use of EDTA with citrate buffer in the presence of PABA. It is preferred in such combinations that citrate, PABA and EDTA as appropriate will be present in the ranges of concentration and preferred ranges of concentration indicated herein. Preferred concentrations for EDTA for use in any aspect of the present invention, including in combination with other agents is around 0.02 to 200 mM, preferably 0.2 to 20 mM and most preferably 0.05 to 8 mM.

In a further embodiment applicable to all compatible aspects of the invention, the tissue-targeting complex may be dissolved in a suitable buffer containing at least one polysorbate (PEG grafted sorbitan fatty-acid ester). Preferred polysorbates include Polysorbate 80 (Polyoxyethylene (20) sorbitan monooleate), Polysorbate 60 (Polyoxyethylene (20) sorbitan monostearate), Polysorbate 40 (Polyoxyethylene (20) sorbitan monopalmitate), Polysorbate 80 (Polyoxyethylene (20) sorbitan monolaurate) and mixtures thereof. Polysorbate 80 (P80) is a most preferred polysorbate. Preferred concentrations for polysorbate (especially preferred polysorbates as indicated herein) for use in any aspect of the present invention, including in combination with other agents is around 0.001 to 10% w/v, preferably 0.01 to 1% w/v and most preferably 0.02 to 0.5 w/v.

Although PABA has been previously described as a radiostabilizer (see U.S. Pat. No. 4,880,615 A) a positive effect of PABA in the present invention was observed on the non-radioactive conjugate on storage. This stabilising effect in the absence of radiolysis constitutes a particularly surprising advantage because the synthesis of the tissue-targeting chelator will typically take place significantly before contacting with the thorium ion. Thus, the tissue-targeting chelator may be generated 1 hour to 3 years prior to contact with the thorium ion and will preferably be stored in contact with PABA during at least a part of that period. That is to say, steps a) and b) of the present invention may take place 1 hour to 3 years before step c) and between steps b) and c), the tissue-targeting chelator may be stored in contact with PABA, particularly in a buffer, such as a citrate buffer and optionally with EDTA and/or a polysorbate. All materials preferably being the type and concentrations indicated herein. PABA is thus a highly preferred component of the formulations of the invention and can result in long term stability for the tissue-targeting chelator and/or for the tissue-targeting thorium complex. FIG. 1 illustrates the effect of PABA in the present system.

The use of citrate buffer as described herein provides a further surprising advantage with regard to the stability of the tissue-targeting thorium complex in the formulations of the present invention. An irradiation study on the effect of buffer-solutions on hydrogen peroxide generation was carried out by the present inventors with unexpected results. Hydrogen peroxide is known to form as a result of water radiolysis and contributes to chemical modification of protein conjugates in solution. Hydrogen peroxide generation therefore has an undesirable effect on the purity and stability of the product. FIGS. 2a-2c show the surprising observation that lower levels of hydrogen peroxide were measured in the antibody HOPO conjugate solutions of this invention irradiated with Co-60 (10 kGy) in citrate buffer compared to all other buffers tested. Thus, the formulations of the present invention will preferably comprising citrate buffer as described herein.

The present inventors have additionally established a further surprising finding relating to the combined effect of certain components in the formulations of this invention. This relates again to the stability of the radiolabelled conjugate. The purpose of the study was to assess the stability of ²²⁷Th-AGC1118 conjugate (see below) during storage. The binding IRF assay was conducted using ²²⁷Th-AGC1118 at a specific activity of around 8000 Bq/μg. Five different storage solutions for the ²²⁷Th-AGC1118 were prepared, using 30 or 100 mM citrate buffer, or 30 mM citrate buffer added either 0.02, 0.2 or 2 mg/mL of pABA, pH 5.5. FIG. 3 shows the significant positive effect on radiostability of the formulations of this invention, particularly when combined with citrate and/or PABA in the ranges indicated herein. Citrate having been found in the above-described study to be the most effective buffer, it was surprising to find that this effect was improved still further by the addition of PABA.

A key component of the methods, complexes and formulations of the present invention is the octadentate chelator moiety. The most relevant previous work on complexation of thorium ions with hydroxypyridinone ligands was published as WO2011/098611 and discloses the relative ease of generation of thorium ions complexed with octadentate HOPO-containing ligands.

Previously known chelators for thorium also include the polyaminopolyacid chelators which comprise a linear, cyclic or branched polyazaalkane backbone with acidic (e.g. carboxyalkyl) groups attached at backbone nitrogens. Examples of such chelators include DOTA derivatives such as p-isothiocyanatobenzyl-1,4,7,10-tetraazacyclododecane-1,4,7,10-tetraacetic acid (p-SCN-Bz-DOTA) and DTPA derivatives such as p-isothiocyanatobenzyl-diethylenetriaminepentaacetic acid (p-SCN-Bz-DTPA), the first being cyclic chelators, the latter linear chelators.

Derivatives of 1,4,7,10-tetraazacyclododecane-1,4,7,10-tetraacetic acid have been previously exemplified, but standard methods cannot easily be used to chelate thorium with DOTA derivatives. Heating of the DOTA derivative with the metal provides the chelate effectively, but often in low yields. There is a tendency for at least a portion of the ligand to irreversibly denature during the procedure. Furthermore, because of its relatively high susceptibility to irreversible denaturation, it is generally necessary to avoid attachment of the targeting moiety until all heating steps are completed. This adds an extra chemical step (with all necessary work-up and separation) which must be carried out during the decay lifetime of the alpha-emitting thorium isotope. Obviously it is preferable not to handle alpha-emitting material in this way or to generate corresponding waste to a greater extent than necessary. Furthermore, all time spent preparing the conjugate wastes a proportion of the thorium which will decay during this preparatory period.

A key aspect of the present invention in all respects is the use of an octadentate ligand, particularly an octadentate hydroxypyridinone-containing ligand comprising four HOPO moieties. Such ligands will typically comprise at least four chelating groups each independently having the following substituted pyridine structure (I):

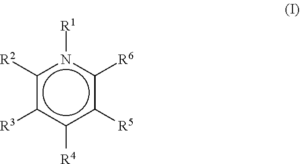

wherein R¹ is an alkyl group such as a C₁ to C₅ straight or branched chain alkyl groups including methyl, ethyl, n- or iso-propyl and n-, sec- iso- or tert-butyl. The preferred R¹ is C₁ to C₃, especially methyl. In one preferred embodiment a methyl substituent present on the nitrogen of all four moieties of formula (I).

Alkyl groups referred to herein will typically be straight or branched chain C₁ to C₈ alkyl groups such as methyl, ethyl, n- or iso-propy, n-, iso- tert- or sec-butyl and so forth.

In certain previous disclosures, such as WO2013/167756, WO2013/167755 and WO2013/167754 the group corresponding to R¹ has primarily been a solubilising group such as hydroxy or hydroxyalkyl (e.g. —CH₂OH, —CH₂—CH₂OH, —CH₂—CH₂—CH₂OH etc). This has certain advantages in terms of higher solubility, but such chelators are difficult to join to targeting moieties using amide bonds because of the reactivity at the R¹ position. In the present invention, therefore, R¹ is generally not hydroxyl or hydroxyalkyl.

In formula (I), groups R² to R⁶ may each independently be selected from H, OH, ═O, a coupling moiety and a linker moiety. Preferably, exactly one of groups R² to R⁶ will be ═O and exactly one of groups R² to R⁶ will be OH. The remaining three of groups R² to R⁶ may be H but at least one of R² to R⁶ will be a linker moiety and/or coupling moiety. The coupling moiety is described herein below but terminates in a carboxylic acid for attachment by an amide bond to the targeting moiety. Such coupling moiety may attach directly to the ring at one of groups R² to R⁶ but will more preferably attach to the linking moietly, which will itself constitute one of groups R² to R⁶.

N-substituted 3,2-HOPO moieties are highly preferred as HOPO groups of the present invention and in one embodiment, all four complexing moieties of the octadentate ligand may be 3,2-HOPO moieties.

Suitable chelating moieties may be formed by methods known in the art, including the methods described in U.S. Pat. No. 5,624,901 (e.g. examples 1 and 2) and WO2008/063721 (both incorporated herein by reference).

Preferred chelating groups include those of formula (II) below:

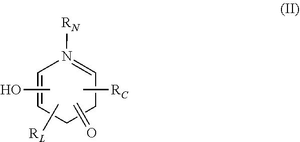

In the above formula (II), the ═O moiety represents an oxo-group attached to any carbon of the pyridine ring, the —OH represents a hydroxy moiety attached to any carbon of the pyridine ring and the —R_{L} represents a linker moiety which attaches the hydroxypyridinone moiety to other complexing moieties so as to form the overall octadentate ligand. Any linker moiety described herein is suitable as R_{L} including short hydrocarbyl groups, such as C₁ to C₈ hydrocarbyl, including C₁ to C₈ alkyl, alkenyl or alkynyl group, including methyl, ethyl, propyl, butyl, pentyl and/or hexyl groups of all topologies. R_{L} may join the ring of formula (II) at any carbon of the pyridine ring. The R_{L} groups may then in turn bond directly to another chelating moiety, to another linker group and/or to a central atom or group, such as a ring or other template (as described herein). The linkers, chelating groups and optional template moieties are selected so as to form an appropriate octadentate ligand.

R_{C} represents a coupling moiety, as discussed below. Suitable moieties include hydrocarbyl groups such as alkyl or akenyl groups terminating in a carboxylic acid group. It has been established by the present inventors that use of a carboxylic acid linking moiety to form an amide, such as by the methods of the present invention, provides a more stable conjugation between the chelator and the tissue-targeting moiety.

In one preferred embodiment the —OH and ═O moieties of formula II reside on neighbouring atoms of the pyridine ring, such that 2,3-, 3,2-; 4,3-; and 3,4-hydroxypyridinone derivatives are all highly suitable. Group R_{N} is a methyl substituent.

In one preferred embodiment, four 3,2-hydroxypyridinone moieties are present in the octadentate ligand structure.

More preferred chelating groups are those of formula (IIa):

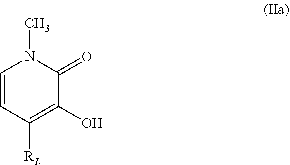

As used herein, the term “linker moiety” (R_{L} in formula (II) and formula (IIa)) is used to indicate a chemical entity which serves to join at least two chelating groups in the octadentate ligands, which form a key component in various aspects of the invention. Linker moieties may also join to the coupling moiety which serves to couple the octadentate ligand portion to the tissue targeting moiety. Typically, each chelating group (e.g. those of formula (I) and/or (II) and/or (IIa) above) will be bi-dentate and so four HOPO chelating groups will typically be present in the ligand. Such chelating groups are joined to each other by means of their linker moieties and are coupled to the tissue-targeting moiety (in the method of the present invention) by means of a coupling moiety. Thus, a linker moiety (e.g. group R_{L} in formula (II)) may be shared between more than one chelating group of formula (I) and/or (II). The linker moieties may also serve as the point of attachment between the complexing part of the octadentate ligand and the targeting moiety. In such a case, at least one linker moiety will join to a coupling moiety (R_{C} in formula (II)). Suitable linker moieties include short hydrocarbyl groups, such as C₁ to C₁₂ hydrocarbyl, including C₁ to C₁₂ alkyl, alkenyl or alkynyl group, including methyl, ethyl, propyl, butyl, pentyl and/or hexyl groups of all topologies. Other groups which may be comprised in the linker moieties (R_{L}) include any suitably robust functional groups such as aryl groups (e.g. phenyl groups), amides, amines (especially secondary or tertiary) and/or ethers. R_{C} moieties may also comprise alkyl and/or aryl sections and optionally groups such as amine, amide and ether linkages. Generally all components of the coupling moiety will need to be robust to the conditions of storage to which the complex will be subjected. This includes alpha-radiolysis and thus labile functional groups are not preferred.

In one embodiment, the coupling moiety comprises a terminal carboxylic acid, at least one alkyl portion (e.g. a methyl or ethyl portion), at least one amide and at least one aryl portion (e.g. a phenyl group). The coupling moiety may be joined to one or more linker moieties of the octadentate ligand by means of a carbon-carbon bond, an amide, an amine and/or an ether linkage.

In the most preferred embodiment of this invention the coupling moiety (R_{C}) linking the octadentate ligand to the targeting moiety is chosen to be [—CH₂-Ph-N(H)—C(═O)—CH₂—CH₂—C(═O)OH], [—CH₂—CH₂—N(H)—C(═O)—(CH₂—CH₂—O)₁₋₃—CH₂—CH₂—C(═O)OH] or [—[CH₂]₁₋₃—Ar—N(H)—C(═O)—[CH₂]₁₋₅—C(═O)OH], wherein Ar is an aromatic group such as a substituted or unsubstituted phenylene group and Ph is a phenylene group, preferably a para-phenylene group.

Linker moieties may be or comprise any other suitably robust chemical linkages including esters, ethers, amine and/or amide groups. The total number of atoms joining two chelating moieties (counting by the shortest path if more than one path exists) will generally be limited, so as to constrain the chelating moieties in a suitable arrangement for complex formation. Thus, linker moieties will typically be chosen to provide no more than 15 atoms between chelating moieties, preferably, 1 to 12 atoms, and more preferably 1 to 10 atoms between chelating moieties. Where a linker moiety joins two chelating moieties directly, the linker will typically be 1 to 12 atoms in length, preferably 2 to 10 (such as ethyl, propyl, n-butyl etc). Where the linker moiety joins to a central template (see below) then each linker may be shorter with two separate linkers joining the chelating moieties. A linker length of 1 to 8 atoms, preferably 1 to 6 atoms may be preferred in this case (methyl, ethyl and propyl being suitable, as are groups such as these having an ester, ether or amide linkage at one end or both).

In addition to the linker moiety, which primarily serves to link the various chelating groups of the octadentate ligand to each other and/or to a central template, the octadentate ligand further comprises a coupling moiety (R_{C}) with a terminal carboxylic acid. The function of the coupling moiety is to link the octadentate ligand to the targeting moiety through a stable covalent bond, especially an amide. Preferably coupling moieties will be covalently linked to the chelating groups, either by direct covalent attachment to one of the chelating groups or more typically by attachment to a linker moiety or template. Should two or more coupling moieties be used, each can be attached to any of the available sites such as on any template, linker or chelating group.

In one embodiment, the coupling moiety may have the structure:

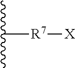

wherein R⁷ is a bridging moiety, which is a member selected from substituted or unsubstituted alkyl, substituted or unsubstituted heteroalkyl, substituted or unsubstituted heterocycloalkyl, substituted or unsubstituted aryl and substituted or unsubstituted heteroaryl; and X is a targeting moiety joined by an amide or a carboxylic acid or equivalent functional group. The preferred bridging moieties include all those groups indicated herein as suitable linker moieties.

Preferred targeting moieties include all of those described herein and preferred reactive X groups include any group capable of acting as a “carboxylic acid” in forming an amide covalent linkage to a targeting moiety, including, for example, —COOH, —SH, —NHR and groups, where the R of NHR may be H or any of the short hydrocarbyl groups described herein. Highly preferred groups for attachment onto the targeting moiety include the epsilon-amines of lysine residues. Non-limiting examples of suitable reactive X groups, include N-hydroxysuccimidylesters, imidoesters, acylhalides, N-maleimides, and alpha-halo acetyl.

In one preferred embodiment of this invention the bridging moiety R⁷ is selected to be substituted aryl and the coupling moiety (R_{C}) linking the octadentate ligand to the targeting moiety is chosen to be [—C(═O)—CH₂CH₂—X-] whereby the free carbon/late group on the HOPO ligand is activated in situ in the form of an N-hydroxysuccinimide ester in aqueous solution immediately prior to conjugation to the targeting moiety.

The coupling moiety is preferably attached, so that the resulting coupled octadentate ligand will be able to undergo formation of stable metal ion complexes. The coupling moiety will thus preferably link to the linker, template or chelating moiety at a site which does not significantly interfere with the complexation. Such a site will preferably be on the linker or template, more preferably at a position distant from the surface binding to the target.

Each moiety of formula (I) or (II) or (IIa) in the octadentate ligand may be joined to the remainder of the ligand by any appropriate linker group as discussed herein and in any appropriate topology. For example, four groups of formula (I) and/or (II) and/or (IIa) may be joined by their linker groups to a backbone so as to form a linear ligand, or may be bridged by linker groups to form an “oligomer” type structure, which may be linear or cyclic. Alternatively, the ligand moieties of formulae (I) and/or (II) and/or (IIa) may be joined in a “cross” or “star” topography to a central atom or group, each by a linker (e.g. “R_{L}” moiety). Linker (R_{L}) moieties may join solely through carbon-carbon bonds, or may attach to each other, to other chelating groups, to a backbone, template, coupling moiety or other linker by any appropriately robust functionality including an amine, amide, ether or thio-ether bond.

A “stellar” arrangement is indicated in formula (III) below:

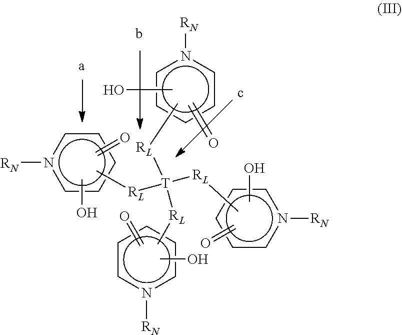

Wherein all groups and positions are as indicated above and “T” is additionally a central atom or template group, such as a carbon atom, hydrocarbyl chain (such as any of those described herein above), aliphatic or aromatic ring (including heterocyclic rings) or fused ring system. The most basic template would be a single carbon, which would then attach to each of the chelating moieties by their linking groups. Longer chains, such as ethyl or propyl are equally viable with two chelating moieties attaching to each end of the template. Evidently, any suitably robust linkage may be used in joining the template and linker moieties including carbon-carbon bonds, ester, ether, amine, amide, thio-ether or disulphide bonds.

Evidently, in the structures of formula (II), (III), (IV) and (IVb), those positions of the pyridine ring(s) which are not otherwise substituted (e.g by a linker or coupling moiety) may carry substituents described for R¹ to R⁵ in formula (I), as appropriate. In particular, small alkyl substituents, such as methyl, ethyl or propyl groups may be present at any position.

The octadentate ligand will additionally comprise at least one coupling moiety as described above. This may be any suitable structure including any of those indicated herein and will terminate with the targeting moiety, in the final complexes or in a carboxylic acid in the methods of the present invention.

The coupling moiety may attach to any suitable point of the linker, template or chelating moiety, such as at points a, b and/or c as indicated in formula (III). The attachment of the coupling moiety may be by any suitably robust linkage such as carbon-carbon bonds, ester, ether, amine, amide, thio-ether or disulphide bonds. Similarly, groups capable of forming any such linkages to the targeting moiety are suitable for the functional end of the coupling moiety and that moiety will terminate with such groups when attached to the targeting part.

An alternative, “backbone” type structure is indicated below in formula (IV)

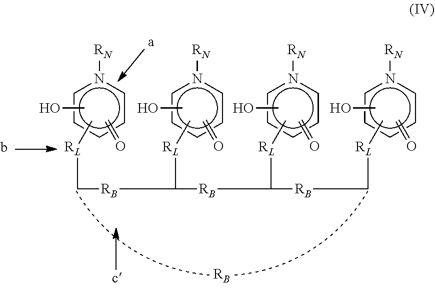

Wherein all groups and positions are as indicated above and “R_{B}” is additionally a backbone moiety, which will typically be of similar structure and function to any of the linker moieties indicated herein, and thus any definition of a linker moiety may be taken to apply to the backbone moiety where context allow. Suitable backbone moieties will form a scaffold upon which the chelating moieties are attached by means of their linker groups. Usually three or four backbone moieties are required. Typically this will be three for a linear backbone or four if the backbone is cyclised. Particularly preferred backbone moieties include short hydrocarbon chains (such as those described herein) optionally having a heteroatom or functional moiety at one or both ends. Amine and amide groups are particularly suitable in this respect.

The coupling moiety may attach to any suitable point of the linker, backbone or chelating moiety, such as at points a, b and/or c′ as indicated in formula (IV). The attachment of the coupling moiety may be by any suitably robust linkage such as carbon-carbon bonds, ester, ether, amine, amide, thio-ether or disulphide bonds. Similarly, groups capable of forming any such linkages to the targeting moiety are suitable for the functional end of the coupling moiety and that moiety will terminate with such groups when attached to the targeting part.

An example of a “backbone” type octadentate ligand having four 3,2-HOPO chelating moieties attached to a backbone by amide linker groups would be formula (V) as follows:

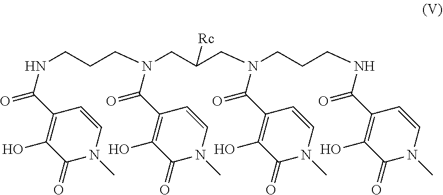

Evidently, a coupling moiety R_{C} may be added at any suitable point on this molecule, such as at one of the secondary amine groups or at a branching point on any of the backbone alkyl groups. A preferred site for group R_{C} is shown in formula (V). R_{C} will terminate in a carboxylic acid, or will be joined by means of an amide linkage to the tissue-targeting moiety in appropriate aspects of the invention. All small alkyl groups such as the backbone propylene or the n-substituting ethylene groups may be substituted with other small alkylenes such as any of those described herein (methylene, ethylene, propylene, and butylene being highly suitable among those).

Exemplary “templated” octadentate ligands, each having four 3,2-HOPO chelating moieties linked by ethyl amide groups to ethyl and propyl diamine respectively would be formula (VI) as follows:

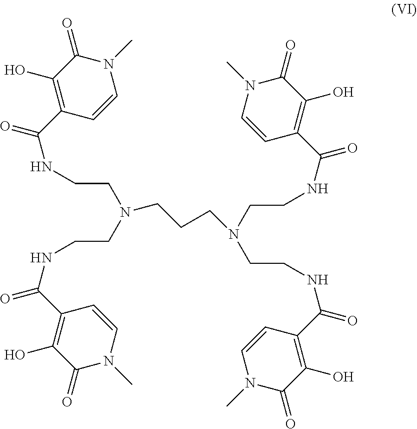 [SMILES: Cn1ccc(C(=O)NCCN(CCCN(CCNC(=O)c2ccn(C)c(=O)c2O)CCNC(=O)c2ccn(C)c(=O)c2O)CCNC(=O)c2ccn(C)c(=O)c2O)c(O)c1=O]

Evidently, any of the alkylene groups, shown in formula (VI) as ethylene moieties may be independently substituted with other small alkylene groups such as methylene, propylene or n-butylene. It is beneficial that symmetry be retained so the central propylene C₃ chain is preferred while the other ethylene groups remain, or the two ethylenes linking the HOPO moieties to one or both central tertiary amines may be replaced with methylene or propylene.

Formula (VIb) shows a possible position for coupling moiety R_{C}, which will be present in formula (VI) at any appropriate position, such as a —CH— group.

As indicated above, the octadentate ligand will typically include a coupling moiety which may join to the remainder of the ligand at any point. A suitable point for coupling moiety attachment is shown below in formula (VIb):

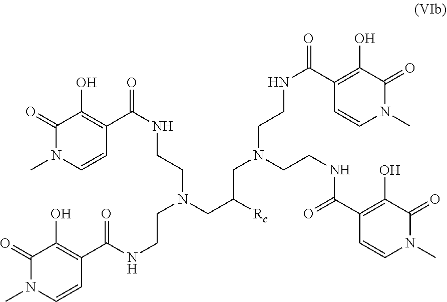

wherein R_{C} is any suitable coupling moiety, particularly for attachment to a tissue targeting group via an amide group. A short hydrocarbyl group such as a C₁ to C₈ cyclic, branched or straight chain aromatic or aliphatic group terminating in an acid or equivalent active group for formation of an amide to the tissue targeting moiety is highly suitable as group R_{C} in formula (VIb) and herein throughout.

Exemplary templates also include others whereby the coupling group R_{C} is covalently linked to a nitrogen atom in the amino backbone as shown in formula (VII).

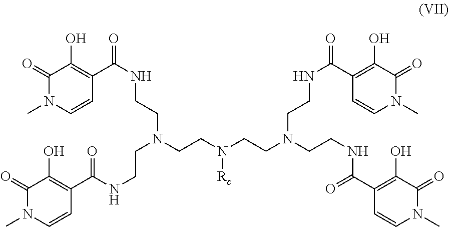

Highly preferred octadentate ligands showing suitable sites for ligand attachment include those of formulae (VIII) and (IX) below:

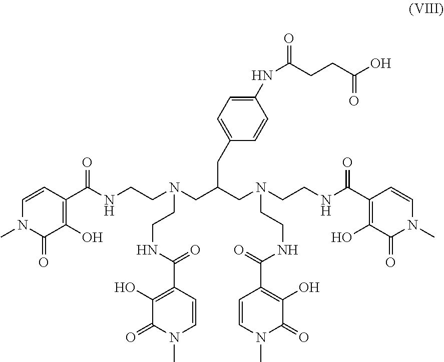

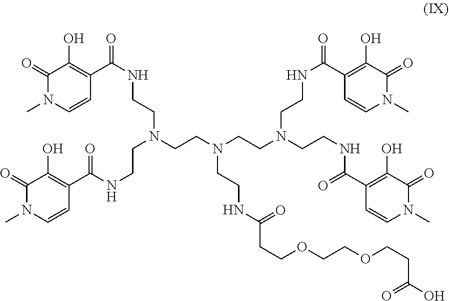 [SMILES: Cn1ccc(C(=O)NCCN(CCNC(=O)c2ccn(C)c(=O)c2O)CCN(CCNC(=O)CCOCCOCCC(=O)O)CCN(CCNC(=O)c2ccn(C)c(=O)c2O)CCNC(=O)c2ccn(C)c(=O)c2O)c(O)c1=O]

The synthesis of compound (VIII) is described herein below and follows the synthetic route described herein below.

AGC0019, and compounds of formulae (VI), (VIb), (VII), (VIII) and (IX) form preferred octadentate chelators having linker moieties terminating in carboxylic acid groups. The octadentate ligands shown in those structures and the linker moieties shown also form preferred examples of their type and may be combined in any combination. Such combinations will be evident to the skilled worker.

Step a) of the methods of the present invention may be carried out by any suitable synthetic route. Typically this will involve linking four HOPO moieties (such as those of formulae (I) and/or (II) and or (IIa)) by means of a linking group to a coupling moiety, optionally by means of a template. All of these groups are described herein and preferred embodiments are equally preferred in this context. Coupling between HOPO moieties, linkers, coupling moiety and optionally template will typically be by means of a robust group such as an amide, amine, ether or carbon-carbon bond. Methods for synthesis of such bonds and any necessary protecting strategies are well known in the art of synthetic chemistry. Some specific examples of synthetic methods are given below in the following Examples. Such methods provide specific examples, but the synthetic methods illustrated therein will also be usable in a general context by those of skill in the art. The methods illustrated in the Examples are therefore intended also as general disclosures applicable to all aspects and embodiments of the invention where context allows.

It is preferred that the complexes of alpha-emitting thorium and an octadentate ligand in all aspects of the present invention are formed or formable without heating above 60° C. (e.g. without heating above 50° C.), preferably without heating above 38° C. and most preferably without heating above 25° C. (such as in the range 20 to 38° C.). Typical ranges may be, for example 15 to 50° C. or 20 to 40° C. The complexation reaction (part c)) in the methods of the present invention) may be carried out for any reasonable period but this will preferably be between 1 and 120 minutes, preferably between 1 and 60 minutes, and more preferably between 5 and 30 minutes.

It is additionally preferred that the conjugate of the targeting moiety and the octadentate ligand be prepared prior to addition of the alpha-emitting thorium isotope (e.g. ²²⁷Th⁴⁺ ion). The products of the invention are thus preferably formed or formable by complexation of alpha-emitting thorium isotope (e.g. ²²⁷Th⁴⁺ ion) by a conjugate of an octadentate ligand and a tissue-targeting moiety (the tissue-targeting chelator).

Various types of targeting compounds may be linked to thorium (e.g. thorium-227) via an octadentate chelator (comprising a coupling moiety as described herein). The targeting moiety may be selected from known targeting groups, which include monoclonal or polyclonal antibodies, growth factors, peptides, hormones and hormone analogues, folate derivatives, biotin, avidin and streptavidin or analogues thereof. Other possible targeting groups include suitable functionalised RNA, DNA, or fragments thereof (such as aptamer), oligonucleotides, carbohydrates, lipids or compounds made by combining such groups with or without proteins etc. PEG moieties may be included as indicated above, such as to increase the biological retention time and/or reduce the immune stimulation.

Generally, as used herein, the tissue targeting moieties will be “peptides” or “proteins”, being structures formed primarily of an amide backbone between amino-acid components either with or without secondary and tertiary structural features.

The tissue targeting moiety may, in one embodiment, exclude bone-seekers, liposomes and folate conjugated antibodies or antibody fragments.

According to this invention ²²⁷Th may be complexed by targeting complexing agents joined or joinable by an amide linkage to tissue-targeting moieties as described herein. Typically the targeting moieties will have a molecular weight from 100 g/mol to several million g/mol (particularly 100 g/mol to 1 million g/mol), and will preferably have affinity for a disease-related receptor either directly, and/or will comprise a suitable pre-administered binder (e.g. biotin or avidin) bound to a molecule that has been targeted to the disease in advance of administering ²²⁷Th. Suitable targeting moieties include poly- and oligo-peptides, proteins, DNA and RNA fragments, aptamers etc, preferably a protein, e.g. avidin, strepatavidin, a polyclonal or monoclonal antibody (including IgG and IgM type antibodies), or a mixture of proteins or fragments or constructs of protein. Antibodies, antibody constructs, fragments of antibodies (e.g. Fab fragments or any fragment comprising at least one antigen binding region(s)), constructs of fragments (e.g. single chain antibodies) or a mixture thereof are particularly preferred. Suitable fragments particularly include Fab, F(ab′)₂, Fab′ and/or scFv. Antibody constructs may be of any antibody or fragment indicated herein.

In a first targeting embodiment applicable to all aspects of the invention, the specific binder (tissue targeting moiety) may be chosen to target the CD22 receptor. Such a tissue targeting moiety may be a peptide with sequence similarity or identity with at least one sequence as set out below:

Light Chain:
Murine
DIQLTQSPSSLAVSAGENVTMSCKSSQSVLYSANHKNYLAWYQQKPGQSP
Humanised
------------SA-V-DR-----------------------------KA
Murine
KLLIYWASTRESGVPDRFTGSGSGTDFTLTISRVQVEDLAIYYCHQYLSS
Humanised
---------------S--S---------F---SL-P--I-T---------
Murine
(SEQ ID NO: 1)
WTFGGGTKLEIKR
Humanised
(SEQ ID NO: 2)
-------------
Heavy Chain:
Murine
QVQLQESGAELSKPGASVKMSCKASGYTFTSYWLHWIKQRPGQGLEWIG
H′ised1
-----Q----VK---S---V----------------VR-A---------
H′ised2
----VQ----VK---S---V----------------VR-A---------
Murine
YINPRNDYTEYNQNFKDKATLTADKSSSTAYMQLSSLTSEDSAVYYCAR
H′ised1
--------------------I---E-TN----E----R---T-F-F---
H′ised2
--------------------I---E-TN----E----R---T-F-F---
Murine
(SEQ ID NO: 3)
RDITTFYWGQGTTLTVSS
H′ised1
(SEQ ID NO: 4)
-------------V----
H′ised2
(SEQ ID NO: 5)
-------------V----

In the above sequences, “-” in the Humanised (H'ised) sequences indicates that the residue is unchanged from the murine sequence.

In the above sequences (SeqID1-5), the bold regions are believed to be the key specific-binding regions (CDRs), the underlined regions are believed to be of secondary importance in binding and the unemphasised regions are believed to represent structural, rather than specific binding regions.

In all aspects of the invention, the tissue targeting moiety may have a sequence having substantial sequence identity or substantial sequence similarity to at least one or any of those sequences set out in SeqID1-5. Substantial sequence identity/similarity may be taken as having a sequence similarity/identity of at least 80% to the complete sequences and/or at least 90% to the specific binding regions (those regions shown in bold in the above sequences and optionally those sections underlined). Preferable sequence similarity or more preferably identity may be at least 92%, 95%, 97%, 98% or 99% for the bold regions and preferably also for the full sequences. Sequence similarity and/or identity may be determined using the “BestFit” program of the Genetics Computer Group Version 10 software package from the University of Wisconsin. The program uses the local had algorithm of Smith and Waterman with default values: Gap creation penalty=8, Gap extension penalty=2, Average match=2.912, average mismatch 2.003.

A tissue targeting moiety may comprise more than one peptide sequence, in which case at least one, and preferably all sequences may (independently) conform to the above-described sequence similarity and preferably sequence identity with any of SeqID1-5.

A tissue targeting moiety may have binding affinity for CD22 and in one embodiment may also have a sequence with up to about 40 variations for the full domains (preferably 0 to 30 variations). Variants may be by insertion, deletion and/or substitution and may be contiguous or non-contiguous with respect to SeqID1-5. Substitutions or insertions will typically be by means of at least one of the 20 amino acids of the genetic code and substitutions will most generally be conservative substitutions.

In a second targeting embodiment applicable to all aspects of the invention, the specific binder (tissue targeting moiety) may be chosen to target the CD33 receptor. Such a tissue targeting moiety may be a monoclonal antibody and may be selected to be lintuzumab or lintuzumab with an extra lysine residue at the C-terminus.

In a third targeting embodiment applicable to all aspects of the invention, the specific binder (tissue targeting moiety) may be chosen to target the HER-2 antigen. The tissue targeting moiety may be a monoclonal antibody and is preferably trastuzumab.

Other suitable antibody sequences for targeting of FGFR2, Mesothelin and PSMA are exemplified in the example section. However it should be obvious to one skilled in the art that any protein format known to target a disease specific target which contains a lysine residue in the sequence would be a candidate for the methods of this invention and correspondingly applicable to all other aspects.

With regard to the alpha-emitting thorium component, it is a key recent finding that certain alpha-radioactive thorium isotopes (e.g. ²²⁷Th) may be administered in an amount that is both therapeutically effective and does not generate intolerable myelotoxicity. Thorium-227 (²²⁷Th) is the preferred thorium isotope in all aspects of the present invention. As used herein, the term “acceptably non-myelotoxic” is used to indicate that, most importantly, the amount of radium-223 generated by decay of the administered thorium-227 radioisotope is generally not sufficient to be directly lethal to the subject. It will be clear to the skilled worker, however, that the amount of marrow damage (and the probability of a lethal reaction) which will be an acceptable side-effect of such treatment will vary significantly with the type of disease being treated, the goals of the treatment regimen, and the prognosis for the subject. Although the preferred subjects for the present invention are humans, other mammals, particularly companion animals such as dogs, will benefit from the use of the invention and the level of acceptable marrow damage may also reflect the species of the subject. The level of marrow damage acceptable will generally be greater in the treatment of malignant disease than for non-malignant disease. One well known measure of the level of myelotoxicity is the neutrophil cell count and, in the present invention, an acceptably non-myelotoxic amount of ²²³Ra will typically be an amount controlled such that the neutrophil fraction at its lowest point (nadir) is no less than 10% of the count prior to treatment. Preferably, the acceptably non-myelotoxic amount of ²²³Ra will be an amount such that the neutrophil cell fraction is at least 20% at nadir and more preferably at least 30%. A nadir neutrophil cell fraction of at least 40% is most preferred.

In addition, radioactive thorium (e.g. ²²⁷Th)) containing compounds may be used in high dose regimens where the myelotoxicity of the generated radium (e.g. ²²³Ra) would normally be intolerable when stem cell support or a comparable recovery method is included. In such cases, the neutrophil cell count may be reduced to below 10% at nadir and exceptionally will be reduced to 5% or if necessary below 5%, providing suitable precautions are taken and subsequent stem cell support is given. Such techniques are well known in the art.

A thorium isotope of particular interest in the present invention is thorium-227, and thorium-227 is the preferred isotope for all references to thorium herein where context allows. Thorium-227 is relatively easy to produce and can be prepared indirectly from neutron irradiated ²²⁶Ra, which will contain the mother nuclide of ²²⁷Th, i.e. ²²⁷AC (T_{1/2}=22 years). Actinium-227 can quite easily be separated from the ²²⁶Ra target and used as a generator for ²²⁷Th. This process can be scaled to industrial scale if necessary, and hence the supply problem seen with most other alpha-emitters considered candidates for molecular targeted radiotherapy can be avoided.

Thorium-227 decays via radium-223. In this case the primary daughter has a half-life of 11.4 days. From a pure ²²⁷Th source, only moderate amounts of radium are produced during the first few days. However, the potential toxicity of ²²³Ra is higher than that of ²²⁷Th since the emission from ²²³Ra of an alpha particle is followed within minutes by three further alpha particles from the short-lived daughters (see Table 2 below which sets out the decay series for thorium-227).

TABLE 2

| | Decay | Mean particle | | |
|---|---|---|---|---|
| Nuclide | mode | energy (MeV) | Half-life | |
| ²²⁷Th | α | 6.02 | 18.72 | days |
| ²²³Ra | α | 5.78 | 11.43 | days |
| ²¹⁹Rn | α | 6.88 | 3.96 | seconds |
| ²¹⁵Po | α | 7.53 | 1.78 | ms |
| ²¹¹Pb | β | 0.45 | 36.1 | minutes |
| ²¹¹Bi | α | 6.67 | 2.17 | minutes |
| ²⁰⁷Tl | β | 1.42 | 4.77 | minutes |

| | |
|---|---|
| ²⁰⁷Pb | Stable |

Partly because it generates potentially harmful decay products, thorium-227 (T_{1/2}=18.7 days) has not been widely considered for alpha particle therapy.

So as to distinguish from thorium complexes of the most abundant naturally occurring thorium isotope, i.e. thorium-232 (half-life 1010 years and effectively non-radioactive), it should be understood that the thorium complexes and the compositions thereof claimed herein include the alpha-emitting thorium radioisotope (i.e. at least one isotope of thorium with a half-life of less than 103 years, e.g. thorium-227) at greater than natural relative abundance, e.g. at least 20% greater. This need not affect the definition of the method of the invention where a therapeutically effective amount of a radioactive thorium, such as thorium-227 is explicitly required, but will preferably be the case in all aspects.

In all aspects of the invention, it is preferable that the alpha-emitting thorium ion is an ion of thorium-227. The 4+ ion of thorium is a preferable ion for use in the complexes of the present invention. Correspondingly, the 4+ ion of thorium-227 is highly preferred.

Thorium-227 may be administered in amounts sufficient to provide desirable therapeutic effects without generating so much radium-223 as to cause intolerable bone marrow suppression. It is desirable to maintain the daughter isotopes in the targeted region so that further therapeutic effects may be derived from their decay. However, it is not necessary to maintain control of the thorium decay products in order to have a useful therapeutic effect without inducing unacceptable myelotoxicity.

Assuming the tumour cell killing effect will be mainly from thorium-227 and not from its daughters, the likely therapeutic dose of this isotope can be established by comparison with other alpha emitters. For example, for astatine-211, therapeutic doses in animals have been typically 2-10 MBq per kg. By correcting for half-life and energy the corresponding dosage for thorium-227 would be at least 36-200 kBq per kg of bodyweight. This would set a lower limit on the amount of ²²⁷Th that could usefully be administered in expectation of a therapeutic effect. This calculation assumes comparable retention of astatine and thorium. Clearly however the 18.7 day half-life of the thorium will most likely result in greater elimination of this isotope before its decay. This calculated dosage should therefore normally be considered to be the minimum effective amount. The therapeutic dose expressed in terms of fully retained ²²⁷Th (i.e. ²²⁷Th which is not eliminated from the body) will typically be at least 18 or 25 kBq/kg, preferably at least 36 kBq/kg and more preferably at least 75 kBq/kg, for example 100 kBq/kg or more. Greater amounts of thorium would be expected to have greater therapeutic effect but cannot be administered if intolerable side effects will result. Equally, if the thorium is administered in a form having a short biological half-life (i.e. the half life before elimination from the body still carrying the thorium), then greater amounts of the radioisotope will be required for a therapeutic effect because much of the thorium will be eliminated before it decays. There will, however, be a corresponding decrease in the amount of radium-223 generated. The above amounts of thorium-227 to be administered when the isotope is fully retained may easily be related to equivalent doses with shorter biological half-lives. Such calculations are well known in the art and given in WO 04/091668 (e.g. in the text an in Examples 1 and 2).

If a radiolabelled compound releases daughter nuclides, it is important to know the fate, if applicable, of any radioactive daughter nuclide(s). With ²²⁷Th, the main daughter product is ²²³Ra, which is under clinical evaluation because of its bone seeking properties. Radium-223 clears blood very rapidly and is either concentrated in the skeleton or excreted via intestinal and renal routes (see Larsen, J Nucl Med 43(5, Supplement): 160P (2002)). Radium-223 released in vivo from ²²⁷Th may therefore not affect healthy soft tissue to a great extent. In the study by Müller in Int. J. Radiat. Biol. 20:233-243 (1971) on the distribution of ²²⁷Th as the dissolved citrate salt, it was found that ²²³Ra generated from ²²⁷Th in soft tissues was readily redistributed to bone or was excreted. The known toxicity of alpha emitting radium, particularly to the bone marrow, is thus an issue with thorium dosages.

It was established for the first time in WO 04/091668 that, in fact, a dose of at least 200 kBq/kg of ²²³Ra can be administered and tolerated in human subjects. These data are presented in that publication. Therefore, it can now be seen that, quite unexpectedly, a therapeutic window does exist in which a therapeutically effective amount of ²²⁷Th (such as greater than 36 kBq/kg) can be administered to a mammalian subject without the expectation that such a subject will suffer an unacceptable risk of serious or even lethal myelotoxicity. Nonetheless, it is extremely important that the best use of this therapeutic window be made and therefore it is essential that the radioactive thorium be quickly and efficiently complexed, and held with very high affinity so that the greatest possible proportion of the dose is delivered to the target site.

The amount of ²²³Ra generated from a ²²⁷Th pharmaceutical will depend on the biological half-life of the radiolabelled compound. The ideal situation would be to use a complex with a rapid tumour uptake, including internalization into tumour cell, strong tumour retention and a short biological half-life in normal tissues. Complexes with less than ideal biological half-life can however be useful as long as the dose of ²²³Ra is maintained within the tolerable level. The amount of radium-223 generated in vivo will be a factor of the amount of thorium administered and the biological retention time of the thorium complex. The amount of radium-223 generated in any particular case can be easily calculated by one of ordinary skill. The maximum administrable amount of ²²⁷Th will be determined by the amount of radium generated in vivo and must be less than the amount that will produce an intolerable level of side effects, particularly myelotoxicity. This amount will generally be less than 300 kBq/kg, particularly less than 200 kBq/kg and more preferably less than 170 kBq/kg (e.g less than 130 kBq/kg). The minimum effective dose will be determined by the cytotoxicity of the thorium, the susceptibility of the diseased tissue to generated alpha irradiation and the degree to which the thorium is efficiently combined, held and delivered by the targeting complex (being the combination of the ligand and the targeting moiety in this case).

In the method of invention, the thorium complex is desirably administered at a thorium-227 dosage of 18 to 400 kBq/kg bodyweight, preferably 36 to 200 kBq/kg, (such as 50 to 200 kBq/kg) more preferably 75 to 170 kBq/kg, especially 100 to 130 kBq/kg. Correspondingly, a single dosage until may comprise around any of these ranges multiplied by a suitable bodyweight, such as 30 to 150 Kg, preferably 40 to 100 Kg (e.g. a range of 540 kBq to 4000 KBq per dose etc). The thorium dosage, the complexing agent and the administration route will moreover desirably be such that the radium-223 dosage generated in vivo is less than 300 kBq/kg, more preferably less than 200 kBq/kg, still more preferably less than 150 kBq/kg, especially less than 100 kBq/kg. Again, this will provide an exposure to ²²³Ra indicated by multiplying these ranges by any of the bodyweights indicated. The above dose levels are preferably the fully retained dose of ²²⁷Th but may be the administered dose taking into account that some ²²⁷Th will be cleared from the body before it decays.

Where the biological half-life of the ²²⁷Th complex is short compared to the physical half-life (e.g. less than 7 days, especially less than 3 days) significantly larger administered doses may be needed to provide the equivalent retained dose. Thus, for example, a fully retained dose of 150 kBq/kg is equivalent to a complex with a 5 day half-life administered at a dose of 711 kBq/kg. The equivalent administered dose for any appropriate retained doses may be calculated from the biological clearance rate of the complex using methods well known in the art.

Since the decay of one ²²⁷Th nucleus provides one ²²³Ra atom, the retention and therapeutic activity of the ²²⁷Th will be directly related to the ²²³Ra dose suffered by the patient. The amount of ²²³Ra generated in any particular situation can be calculated using well known methods.

In a preferred embodiment, the present invention therefore provides a method for the treatment of disease in a mammalian subject (as described herein), said method comprising administering to said subject a therapeutically effective quantity of at least one tissue-targeting thorium complex as described herein.

It is obviously desirable to minimise the exposure of a subject to the ²²³Ra daughter isotope, unless the properties of this are usefully employed. In particular, the amount of radium-223 generated in vivo will typically be greater than 40 kBq/kg, e.g. greater than 60 kBq/Kg. In some cases it will be necessary for the ²²³Ra generated in vivo to be more than 80 kBq/kg, e.g. greater than 100 or 115 kBq/kg.

Thorium-227 labelled conjugates in appropriate carrier solutions may be administered intravenously, intracavitary (e.g. intraperitoneally), subcutaneously, orally or topically, as a single application or in a fractionated application regimen. Preferably the complexes conjugated to a targeting moiety will be administered as solutions by a parenteral (e.g. transcutaneous) route, especially intravenously or by an intracavitary route. Preferably, the compositions of the present invention will be formulated in sterile solution for parenteral administration.

Thorium-227 in the methods and products of the present invention can be used alone or in combination with other treatment modalities including surgery, external beam radiation therapy, chemotherapy, other radionuclides, or tissue temperature adjustment etc. This forms a further, preferred embodiment of the method of the invention and formulations/medicaments may correspondingly comprise at least one additional therapeutically active agent such as another radioactive agent or a chemotherapeutic agent.

In one particularly preferred embodiment the subject is also subjected to stem cell treatment and/or other supportive therapy to reduce the effects of radium-223 induced myelotoxicity.

The thorium (e.g. thorium-227) labelled molecules of the invention may be used for the treatment of cancerous or non-cancerous diseases by targeting disease-related receptors. Typically, such a medical use of ²²⁷Th will be by radioimmunotherapy based on linking ²²⁷Th by a chelator to an antibody, an antibody fragment, or a construct of antibody or antibody fragments for the treatment of cancerous or non-cancerous diseases. The use of ²²⁷Th in methods and pharmaceuticals according to the present invention is particularly suitable for the treatment of any form of cancer including carcinomas, sarcomas, lymphomas and leukemias, especially cancer of the lung, breast, prostate, bladder, kidney, stomach, pancreas, oesophagus, brain, ovary, uterus, oral cancer, colorectal cancer, melanoma, multiple myeloma and non-Hodgkin's lymphoma.

In a further embodiment of the invention, patients with both soft tissue and skeletal disease may be treated both by the ²²⁷Th and by the ²²³Ra generated in vivo by the administered thorium. In this particularly advantageous aspect, an extra therapeutic component to the treatment is derived from the acceptably non-myelotoxic amount of ²²³Ra by the targeting of the skeletal disease. In this therapeutic method, ²²⁷Th is typically utilised to treat primary and/or metastatic cancer of soft tissue by suitable targeting thereto and the ²²³Ra generated from the ²²⁷Th decay is utilised to treat related skeletal disease in the same subject. This skeletal disease may be metastases to the skeleton resulting from a primary soft-tissue cancer, or may be the primary disease where the soft-tissue treatment is to counter a metastatic cancer. Occasionally the soft tissue and skeletal diseases may be unrelated (e.g. the additional treatment of a skeletal disease in a patient with a rheumatological soft-tissue disease).

Conditions which are particularly suitable for treatment in the methods, uses and other aspects of the present invention include neoplastic and hyperplastic diseases such as a carcinoma, sarcoma, myeloma, leukemia, lymphoma or mixed type cancer, including Non-Hodgkin's Lymphoma or B-cell neoplasms, breast, endometrial, gastric, acute myeloid leukemia, prostate or brain, mesothelioma, ovarian, lung or pancreatic cancer

Below are provided some example syntheses. The steps shown in these syntheses will be applicable to many embodiments of the present invention. Step a) for example, may proceed via intermediate AGC0021 shown below in many or all of the embodiments described herein.

### Synthesis of AGC0020 Key Intermediate

### N,N,N′,N′-tetrakis(2-aminoethyl)-2-(4-nitrobenzyl)propane-1,3-diamine

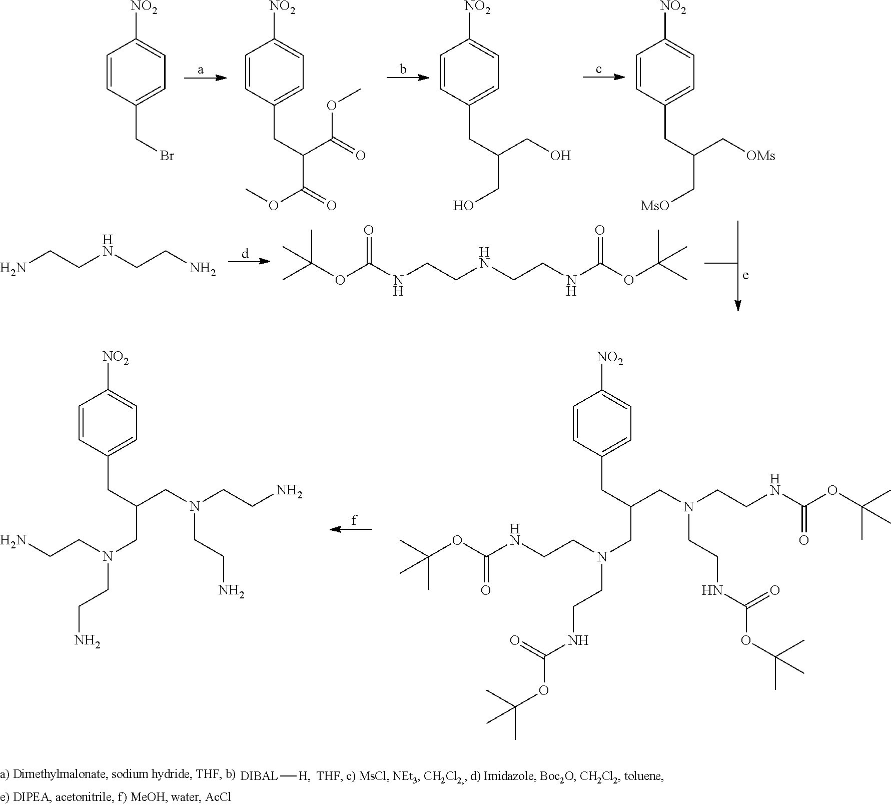

### Synthesis of AGC0021 Key Intermediate

### 3-(benzyloxy)-1-methyl-4-[(2-thioxo-1,3-thiazolidin-3-yl)carbonyl]pyridin-2(1H)-one

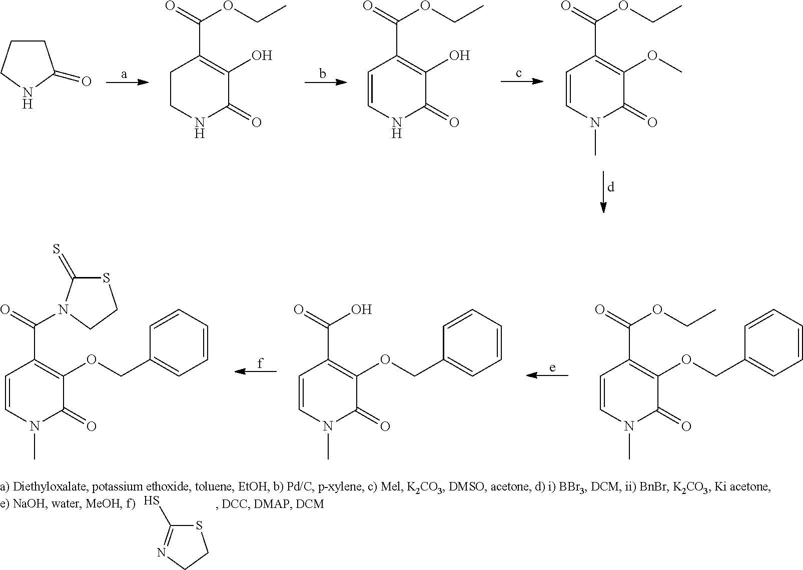

### Synthesis of Chelate of Compound of Formula (VIII)

### 4-{[4-(3-[bis(2-{[(3-hydroxy-1-methyl-2-oxo-1,2-dihydropyridin-4-yl)carbonyl]amino}ethyl)amino]-2-{[bis(2-{[(3-hydroxy-1-methyl-2-oxo-1,2-dihydropyridin-4-yl)carbonyl]amino}ethyl)amino]methyl}propyl)phenyl]amino}-4-oxobutanoic acid

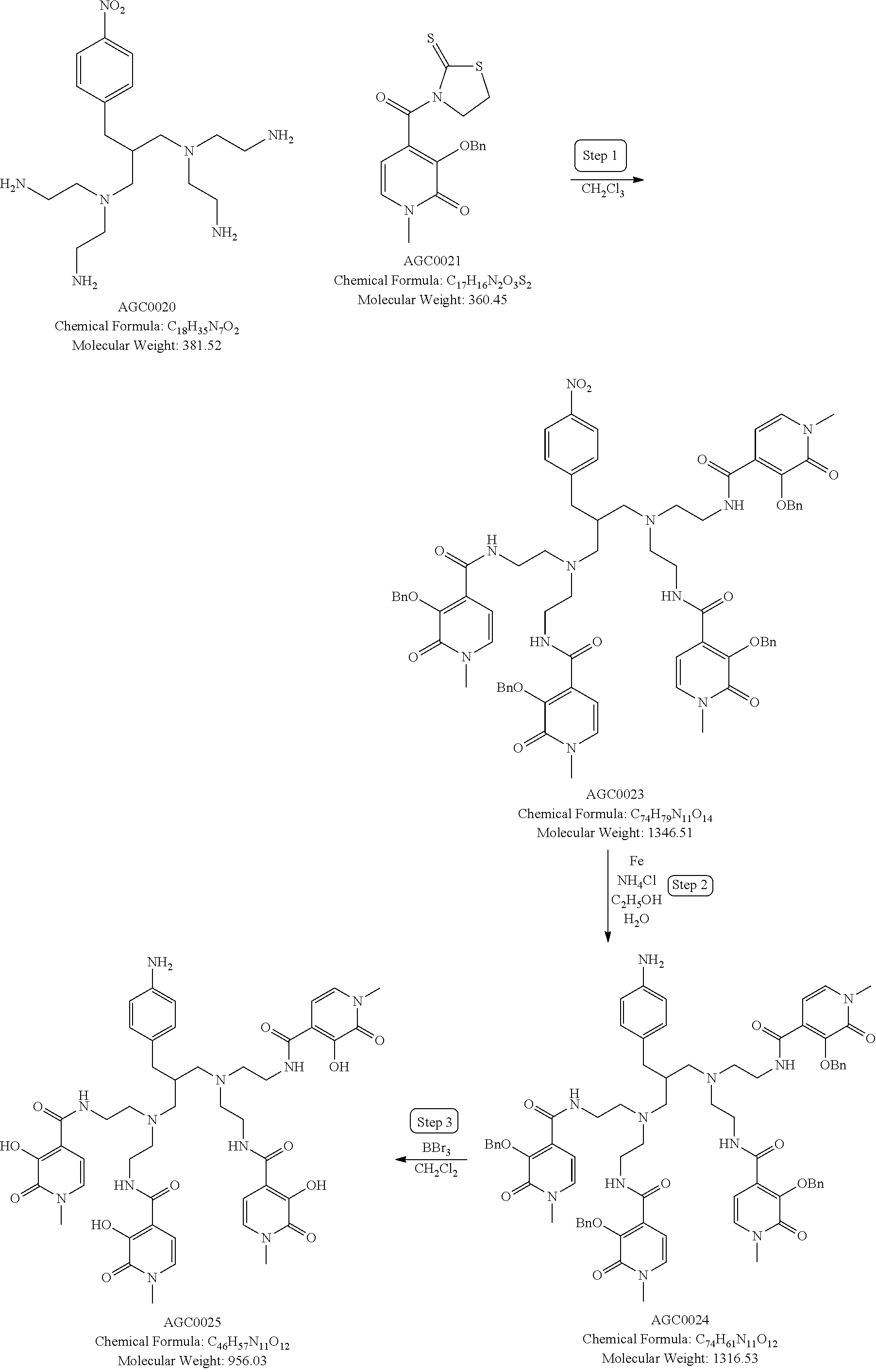

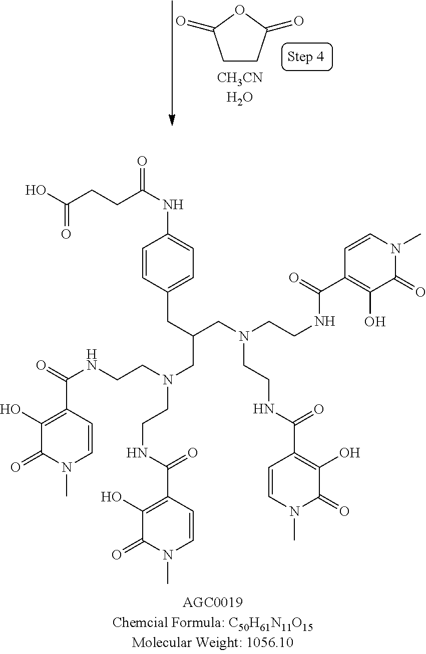 [SMILES: O=C1CCC(=O)O1 ; Cn1ccc(C(=O)NCCN(CCNC(=O)c2ccn(C)c(=O)c2O)CC(Cc2ccc(NC(=O)CCC(=O)O)cc2)CN(CCNC(=O)c2ccn(C)c(=O)c2O)CCNC(=O)c2ccn(C)c(=O)c2O)c(O)c1=O]

In the methods of formation of the complexes of the present invention, it is preferred that the coupling reaction between the octadentate chelator and the tissue targeting moiety be carried out in aqueous solution. This has several advantages. Firstly, it removes the burden on the manufacturer to remove all solvent to below acceptable levels and certify that removal. Secondly it reduces waste and most importantly it speeds production by avoiding a separation or removal step. In the context of the present radiopharmaceuticals, it is important that synthesis be carried out as rapidly as possible since the radioisotope will be decaying at all times and time spent in preparation wastes valuable material and introduces contaminant daughter isotopes.

Suitable aqueous solutions include purified water and buffers such as any of the many buffers well known in the art. Acetate, citrate, phosphate (e.g. PBS) and sulphonate buffers (such as MES) are typical examples of well-known aqueous buffers.

In one embodiment, the method comprises forming a first aqueous solution of octadentate hydroxypyridinone-containing ligand (as described herein throughout) and a second aqueous solution of a tissue targeting moiety (as described herein throughout) and contacting said first and said second aqueous solutions.

Suitable coupling moieties are discussed in detail above and all groups and moieties discussed herein as coupling and/or linking groups may appropriately be used for coupling the targeting moiety to the ligand. Some preferred coupling groups include amide, ester, ether and amine coupling groups. Esters and amides may conveniently be formed by means of generation of an activated ester groups from a carboxylic acid. Such a carboxylic acid may be present on the targeting moiety, on the coupling moiety and/or on the ligand moiety and will typically react with an alcohol or amine to form an ester or amide. Such methods are very well known in the art and may utilise well known activating reagents including N-hydroxy maleimide, carbodiimide and/or azodicarboxylate activating reagents such as DCC, DIC, EDC, DEAD, DIAD etc.

In a preferred embodiment, the octadentate chelator comprising four hydroxypyridinone moieties, substituted in the N-position with a C₁-C₃ alkyl group, and a coupling moiety terminating in a carboxylic acid group may be activated using at least one coupling reagent (such as any of those described herein) and an activating agent such as an N-hydroxysuccinimide (NHS) whereby to form the NHS ester of the octadentate chelator. This activated (e.g. NHS) ester may be separated or used without separation for coupling to any tissue targeting moiety having a free amine group (such as on a lysine side-chain). Other activated esters are well known in the art and may be any ester of an effective leaving group, such as fluorinated groups, tosylates, mesylates, iodide etc. NHS esters are preferred, however.

The coupling reaction is preferably carried out over a comparatively short period and at around ambient temperature. Typical periods for the 1-step or 2-step coupling reaction will be around 1 to 240 minutes, preferably 5 to 120 minutes, more preferably 10 to 60 minutes. Typical temperatures for the coupling reaction will be between 0 and 90° C., preferably between 15 and 50° C., more preferably between 20 and 40° C. Around 25° C. or around 38° C. are appropriate.

Coupling of the octadentate chelator to the targeting moiety will typically be carried out under conditions which do not adversely (or at least not irreversibly) affect the binding ability of the targeting moiety. Since the binders are generally peptide or protein based moieties, this requires comparatively mild conditions to avoid denaturation or loss of secondary/tertiary structure. Aqueous conditions (as discussed herein in all contexts) will be preferred, and it will be desirable to avoid extremes of pH and/or redox. Step b) may thus be carried out at a pH between 3 and 10, preferably between 4 and 9 and more preferably between 4.5 and 8. Conditions which are neutral in terms of redox, or very mildly reducing to avoid oxidation in air may be desirable.

A preferred tissue-targeting chelator applicable to all aspects of the invention is AGC0018 as described herein. Complexes of AGC0018 with ions of ²²⁷Th form a preferred embodiment of the complexes of the invention and corresponding formulations, uses, methods etc. Other preferred embodiments usable in all such aspects of the invention include ²²⁷Th complexes of AGC0019 conjugated to tissue targeting moieties (as described herein) including monoclonal antibodies with binding affinity for any one of CD22 receptor, FGFR2, Mesothelin, HER-2, PSMA or CD33

### BRIEF DESCRIPTION OF THE DRAWINGS

FIG. 1: Data demonstrating the stabilising effect of EDTA/PABA on the non-radioactive antibody conjugate AGC1118 in solution.

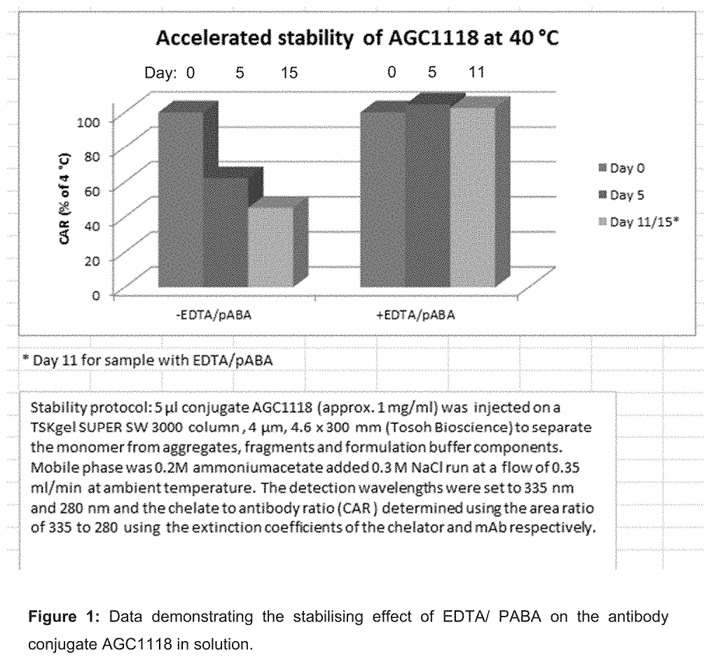

FIGS. 2a-2c: Effect on hydrogen peroxide levels of different buffers containing antibody HOPO conjugates irradiated with 10 kGy of radiation.

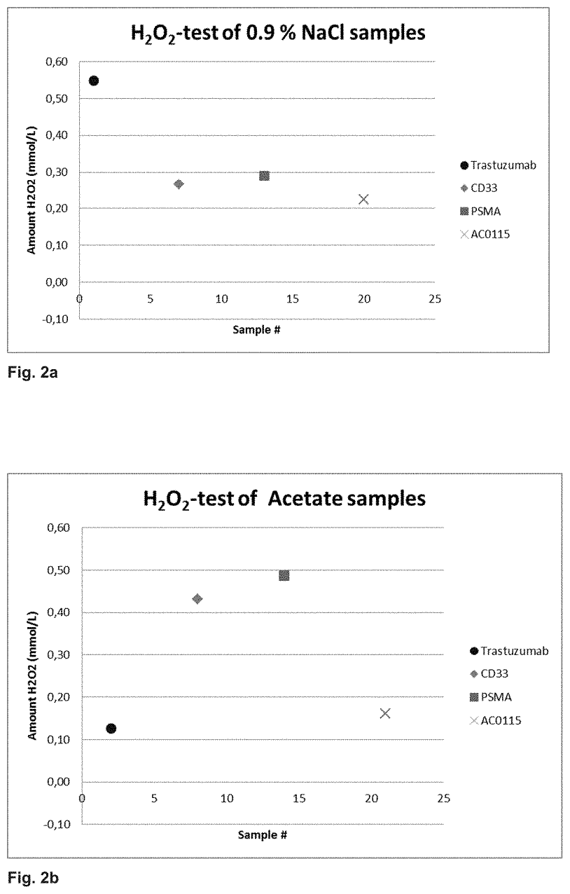

FIG. 3: Radiostabilizing effect of ²²⁷Th-AGC1118 (IRF assay) with a specific activity up to ca 8000 Bq/μg.

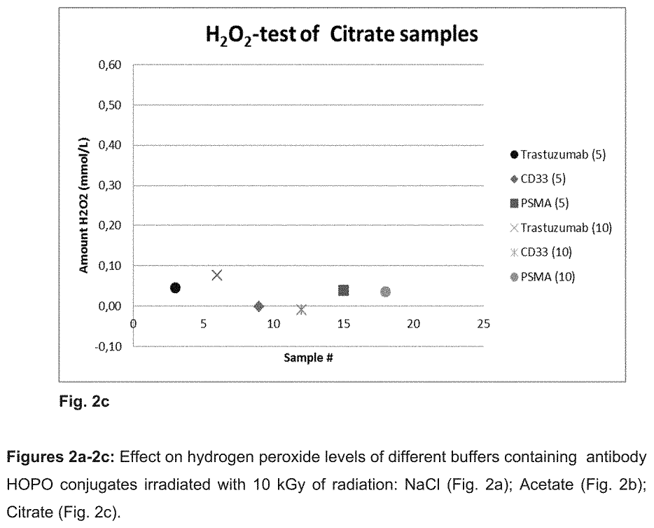

FIG. 4: Cytotoxicity of ²²⁷Th-AGC1118 against Ramos with different total activity (4 hours incubation time) (see Example 3)

FIG. 5: ²²⁷Th-AGC0718 induces target-specific cell killing of CD33-positive cells in vitro (see Example 4)

FIG. 6: Cell cytotoxicity of ²²⁷Th-AGC0118 at high (20 kBq/μg) and low (7.4 kBq/μg) specific activity. Negative control was a low-binding peptide-albumin complex with same dose range, same incubation time and days before readout (see Example 5).

FIG. 7: ²²⁷Th-AGC2518 induces target-specific cell killing of FGFR2-positive cells in vitro (see Example 6).

FIG. 8: ²²⁷Th-AGC2418 induces target-specific cell killing of Mesothelin-positive cells in vitro (see Example 7).

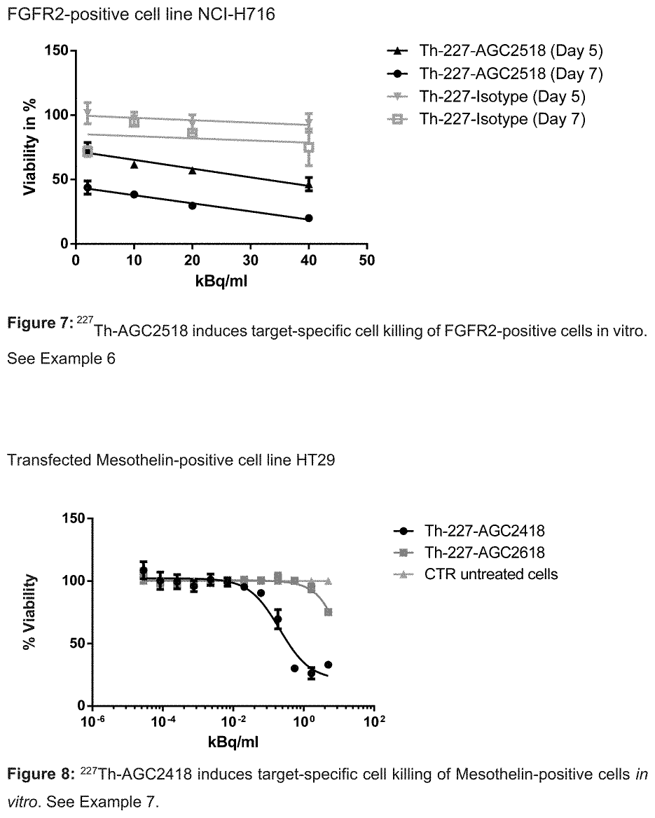

FIG. 9: ²²⁷Th-AGC1018 induces target-specific and dose dependent cell killing of PSMA-positive LNCaP cells in vitro (see Example 9).

The invention will now be illustrated by the following non-limiting examples. All compounds exemplified in the examples form preferred embodiments of the invention (including preferred intermediates and precursors) and may be used individually or in any combination in any aspect where context allows. Thus, for example, each and all of compounds 2 to 4 of Example 2, compound 10 of Example 3 and compound 7 of Example 4 form preferred embodiments of their various types.

### Example 1

### Synthesis of Compound of Formula (VIII)

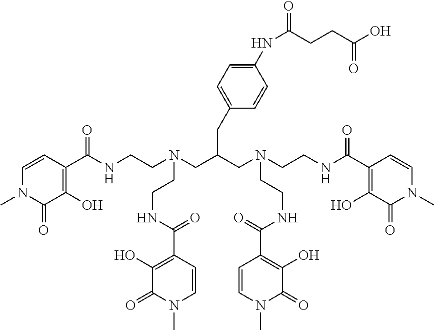

### Example 1 a)

### Synthesis of Dimethyl 2-(4-nitrobenzyl) malonate

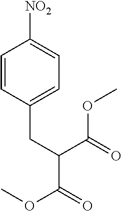 [SMILES: COC(=O)C(Cc1ccc([N+](=O)[O-])cc1)C(=O)OC]

Sodium hydride (60% dispersion, 11.55 g, 289 mmol) was suspended in 450 mL tetrahydrofuran (THF) at 0° C. Dimethyl malonate (40.0 mL, 350 mmol) was added drop wise over approximately 30 minutes. The reaction mixture was stirred for 30 minutes at 0° C. 4-Nitrobenzyl bromide (50.0 g, 231 mmol) dissolved in 150 mL THF was added drop wise over approximately 30 minutes at 0° C., followed by two hours at ambient temperature.

500 mL ethyl acetate (EtOAc) and 250 mL NH₄Cl (aq, sat) was added before the solution was filtered. The phases were separated. The aqueous phase was extracted with 2*250 mL EtOAc. The organic phases were combined, washed with 250 mL brine, dried over Na₂SO₄, filtered and the solvents were removed under reduced pressure.

300 mL heptane and 300 mL methyl tert-butyl ether (MTBE) was added to the residue and heated to 60° C. The solution was filtered. The filtrate was placed in the freezer overnight and filtered. The filter cake was washed with 200 mL heptane and dried under reduced pressure, giving the title compound as an off-white solid.

Yield: 42.03 g, 157.3 mmol, 68%.

1H-NMR (400 MHz, CDCl3): 3.30 (d, 2H, 7.8 Hz), 3.68 (t, 1H, 7.8 Hz), 3.70 (s, 6H), 7.36 (d, 2H, 8.7 Hz), 8.13 (d, 2H, 8.7 Hz).

### Example 1 b)

### Synthesis of 2-(4-Nitrobenzyl)propane-1,3-diol

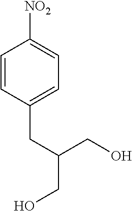 [SMILES: O=[N+]([O-])c1ccc(CC(CO)CO)cc1]

Dimethyl 2-(4-nitrobenzyl) malonate (28.0 g, 104.8 mmol) was dissolved in 560 mL THF at 0° C. Diisobutylaluminium hydride (DIBAL-H) (1M in hexanes, 420 mL, 420 mmol) was added drop wise at 0° C. over approximately 30 minutes. The reaction mixture was stirred for two hours at 0° C.

20 mL water was added drop wise to the reaction mixture at 0° C. 20 mL NaOH (aq, 15%) was added drop wise to the reaction mixture at 0° C. followed by drop wise addition of 20 mL water to the reaction mixture. The mixture was stirred at 0° C. for 20 minutes before addition of approximately 150 g MgSO4. The mixture was stirred at room temperature for 30 minutes before it was filtered on a Büchner funnel. The filter cake was washed with 500 mL EtOAc. The filter cake was removed and stirred with 800 mL EtOAc and 200 mL MeOH for approximately 30 minutes before the solution was filtered. The filtrates were combined and dried under reduced pressure.

DFC on silica using a gradient of EtOAc in heptane, followed by a gradient of MeOH in EtOAc gave the title compound as a pale yellow solid.

Yield: 15.38 g, 72.8 mmol, 69%.

1H-NMR (400 MHz, CDCl3): 1.97-2.13 (m, 3H), 2.79 (d, 2H, 7.6 Hz), 3.60-3.73 (m, 2H), 3.76-3.83 (m, 2H), 7.36 (d, 2H, 8.4 Hz), 8.14 (d, 2H, 8.4 Hz).

### Example 1 c)

### Synthesis of 2-(4-Nitrobenzyl)propane-1,3-diyl dimethanesulfonate

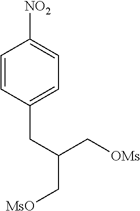 [SMILES: CS(=O)(=O)OCC(COS(C)(=O)=O)Cc1ccc([N+](=O)[O-])cc1]

2-(4-nitrobenzyl)propane-1,3-diol (15.3 g, 72.4 mmol) was dissolved in 150 mL CH₂Cl₂ at 0° C. Triethylamine (23 mL, 165 mmol) was added, followed by methanesulfonyl chloride (12 mL, 155 mmol) drop wise over approximately 15 minutes, followed by stirring at ambient temperature for one hour.

500 mL CH₂Cl₂ was added, and the mixture was washed with 2*250 mL NaHCO₃ (aq, sat), 125 mL HCl (aq, 0.1 M) and 250 mL brine. The organic phase was dried over Na₂SO₄, filtered and dried under reduced pressure, giving the title compound as an orange solid.

Yield: 25.80 g, 70.2 mmol, 97%.

1H-NMR (400 MHz, CDCl3): 2.44-2.58 (m, 1H), 2.87 (d, 2H, 7.7 Hz), 3.03 (s, 6H), 4.17 (dd, 2H, 10.3, 6.0 Hz), 4.26 (dd, 2H, 10.3, 4.4 Hz), 7.38 (d, 2H, 8.6 Hz), 8.19 (d, 2H, 8.6 Hz).

### Example 1 d)

### Synthesis of Di-tert-butyl(azanediylbis(ethane-2,1-diyl))dicarbamate

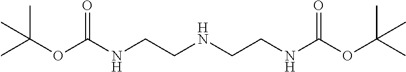 [SMILES: CC(C)(C)OC(=O)NCCNCCNC(=O)OC(C)(C)C]

Imidazole (78.3 g, 1.15 mol) was suspended in 500 mL CH₂Cl₂ at room temperature. Di-tert-butyl dicarbonate (Boc₂O) (262.0 g, 1.2 mol) was added portion wise. The reaction mixture was stirred for one hour at room temperature. The reaction mixture was washed with 3*750 mL water, dried over Na₂SO₄, filtered and the volatiles were removed under reduced pressure.

The residue was dissolved in 250 mL toluene and diethylenetriamine (59.5 mL, 550 mmol) was added. The reaction mixture was stirred for two hours at 60° C.

1 L CH₂Cl₂ was added, and the organic phase was washed with 2*250 mL water. The organic phase was dried over Na₂SO₄, filtered and reduced under reduced pressure.

DFC on silica using a gradient of methanol (MeOH) in CH₂Cl₂ with triethylamine gave the title compound as a colorless solid.

Yield: 102 g, 336 mmol, 61%.

¹H-NMR (400 MHz, CDCl3): 1.41 (s, 18H), 1.58 (bs, 1H), 2.66-2.77 (m, 4H), 3.13-3.26 (m, 4H), 4.96 (bs, 2H).

### Example 1 e)

### Synthesis of Tetra-tert-butyl(((2-(4-nitrobenzyl)propane-1,3-diyl)bis(azanetriyl))tetrakis(ethane-2,1-diyl))tetracarbamate

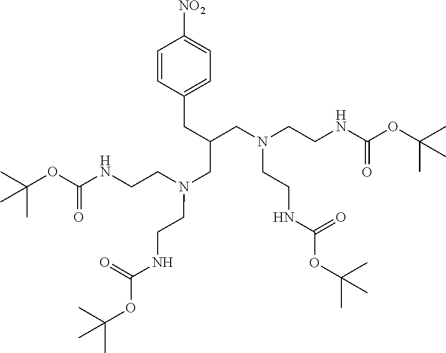 [SMILES: CC(C)(C)OC(=O)NCCN(CCNC(=O)OC(C)(C)C)CC(Cc1ccc([N+](=O)[O-])cc1)CN(CCNC(=O)OC(C)(C)C)CCNC(=O)OC(C)(C)C]

2-(4-Nitrobenzyl)propane-1,3-diyl dimethanesulfonate (26.0 g, 71 mmol) and di-tert-butyl(azanediylbis(ethane-2,1-diyl))dicarbamate (76.0 g, 250 mmol) were dissolved in 700 mL acetonitrile. N,N-diisopropylethylamine (43 mL, 250 mmol) was added. The reaction mixture was stirred for 4 days at reflux.

The volatiles were removed under reduced pressure.

DFC on silica using a gradient of EtOAc in heptane gave the tile compound as pale yellow solid foam.

Yield: 27.2 g, 34.8 mmol, 49%.

¹H-NMR (400 MHz, CDCl3): 1.40 (s, 36H), 1.91-2.17 (m, 3H), 2.27-2.54 (m, 10H), 2.61-2.89 (m, 2H), 2.98-3.26 (m, 8H), 5.26 (bs, 4H), 7.34 (d, 2H, 8.5 Hz), 8.11 (d, 2H, 8.5 Hz).

### Example 1 f)

### Synthesis of N¹,N^{1′}-(2-(4-nitrobenzyl)propane-1,3-diyl)bis(N¹-(2-aminoethyl)ethane-1,2-diamine), AGC0020

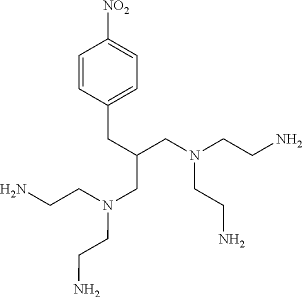 [SMILES: NCCN(CCN)CC(Cc1ccc([N+](=O)[O-])cc1)CN(CCN)CCN]

Tetra-tert-butyl(((2-(4-nitrobenzyl)propane-1,3-diyl)bis(azanetriyl))tetrakis(ethane-2,1-diyl))tetracarbamate (29.0 g, 37.1 mmol) was dissolved in 950 mL MeOH and 50 mL water. Acetyl chloride (50 mL, 0.7 mol) was added drop wise over approximately 20 minutes at 30° C. The reaction mixture was stirred overnight.

The volatiles were removed under reduced pressure and the residue was dissolved in 250 mL water. 500 mL CH₂Cl₂ was added, followed by 175 mL NaOH (aq, 5M, saturated with NaCl). The phases were separated, and the aqueous phase was extracted with 4*250 mL CH₂Cl₂. The organic phases were combined, dried over Na₂SO₄, filtered and dried under reduced pressure, giving the title compound as viscous red brown oil.

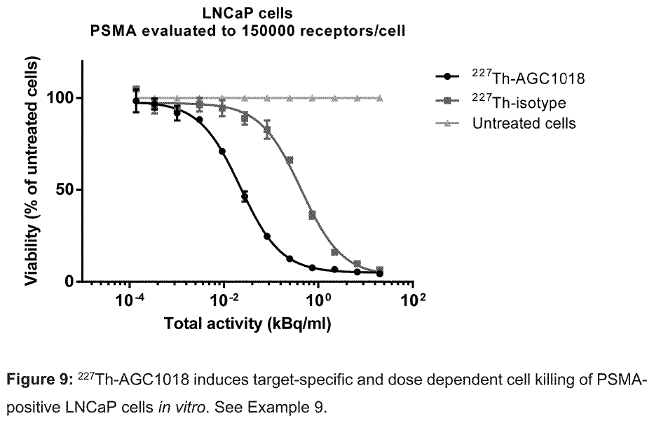

Yield: 11.20 g, 29.3 mmol, 79%. Purity (HPLC FIG. 9): 99.3%.

¹H-NMR (300 MHz, CDCl₃): 1.55 (bs, 8H), 2.03 (dt, 1H, 6.6, 13.3 Hz), 2.15 (dd, 2H, 12.7, 6.6), 2.34-2.47 (m, 10H), 2.64-2.77 (m, 10H), 7.32 (d, 2H, 8.7 Hz), 8.10 (d, 2H, 8.7 Hz).

¹³C-NMR (75 MHz, CDCl₃): 37.9, 38.5, 39.9, 58.0, 58.7, 123.7, 130.0, 146.5, 149.5

### Example 1 g)

### Synthesis of Ethyl 5-hydroxy-6-oxo-1,2,3,6-tetrahydropyridine-4-carboxylate

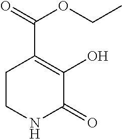

2-pyrrolidinone (76 mL, 1 mol) and diethyl oxalate (140 mL, 1.03 mol) was dissolved in 1 L toluene at room temperature. Potassium ethoxide (EtOK) (24% in EtOH, 415 mL, 1.06 mol) was added, and the reaction mixture was heated to 90° C.

200 mL EtOH was added portion wise during the first hour of the reaction due to thickening of the reaction mixture. The reaction mixture was stirred overnight and cooled to room temperature. 210 mL HCl (5M, aq) was added slowly while stirring.

200 mL brine and 200 mL toluene was added, and the phases were separated.

The aqueous phase was extracted with 2×400 mL CHCl₃. The combined organic phases were dried (Na₂SO₄), filtered and reduced in vacuo. The residue was recrystallized from EtOAc, giving the title compound as a pale yellow solid.

Yield: 132.7 g, 0.72 mol, 72%.

### Example 1 h)

### Synthesis of Ethyl 3-hydroxy-2-oxo-1,2-dihydropyridine-4-carboxylate

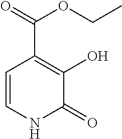

{Ethyl 5-hydroxy-6-oxo-1,2,3,6-tetrahydropyridine-4-carboxylate} (23.00 g, 124.2 mmol) was dissolved in 150 mL p-xylene and Palladium on carbon (10%, 5.75 g) was added. The reaction mixture was stirred at reflux over night. After cooling to room temperature, the reaction mixture was diluted with 300 mL MeOH and filtered through a short pad of Celite®. The pad was washed with 300 mL MeOH. The solvents were removed in vacuo, giving the title compound as a pale red-brownish solid.

Yield: 19.63 g, 107.1 mmol, 86%. MS (ESI, pos): 206.1 [M+Na]⁺, 389.1 [2M+Na]⁺

### Example 1 i)

### Synthesis of Ethyl 3-methoxy-1-methyl-2-oxo-1,2-dihydropyridine-4-carboxylate

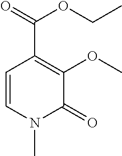

{ethyl 3-hydroxy-2-oxo-1,2-dihydropyridine-4-carboxylate} (119.2 g, 0.65 mol) was dissolved in 600 mL dimethyl sulfoxide (DMSO) and 1.8 L acetone at room temperature. K₂CO₃ (179.7 g, 1.3 mol) was added. Methyl iodide (Mel) (162 mL, 321 mmol) dissolved in 600 mL acetone was added drop wise over approximately 1 hour at room temperature. The reaction mixture was stirred for an additional two hours at room temperature before Mel (162 mL, 2.6 mol) was added. The reaction mixture was stirred at reflux overnight. The reaction mixture was reduced under reduced pressure and 2.5 L EtOAc was added. The mixture was filtered and reduced under reduced pressure. Purification by dry flash chromatography (DFC) on SiO₂ using a gradient of EtOAc in heptane gave the title compound.

Yield: 56.1 g, 210.1 mmol, 32%. MS (ESI, pos): 234.1 [M+Na]⁺, 445.1 [2M+Na]⁺

### Example 1 j)

### Synthesis of Ethyl 3-(benzyloxy)-1-methyl-2-oxo-1,2-dihydropyridine-4-carboxylate

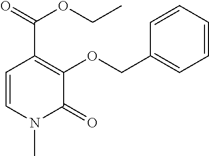 [SMILES: CCOC(=O)c1ccn(C)c(=O)c1OCc1ccccc1]

{ethyl 3-methoxy-1-methyl-2-oxo-1,2-dihydropyridine-4-carboxylate} (5.93 g, 28.1 mmol) was dissolved in 80 mL dichlormethane (DCM) at −78° C. and BBr₃ (5.3 mL, 56.2 mmol) dissolved in 20 mL DCM was added drop wise. The reaction mixture was stirred for 1 hour at −78° C. before heating the reaction to 0° C. The reaction was quenched by drop wise addition of 25 mL tert-butyl methyl ether (tert-BuOMe) and 25 mL MeOH. The volatiles were removed in vacuo. The residue was dissolved in 90 mL DCM and 10 mL MeOH and filtered through a short pad of SiO₂. The pad was washed with 200 mL 10% MeOH in DCM. The volatiles were removed in vacuo. The residue was dissolved in 400 mL acetone. K₂CO₃ (11.65 g, 84.3 mmol), KI (1.39 g, 8.4 mmol) and benzyl bromide (BnBr) (9.2 mL, 84.3 mmol) were added. The reaction mixture was stirred at reflux overnight. The reaction mixture was diluted with 200 mL EtOAc and washed with 3×50 mL water and 50 mL brine. The combined aqueous phases were extracted with 2×50 mL EtOAc. The combined organic phases were dried (Na₂SO₄), filtered, and the volatiles were removed in vacuo and purified by dry flash chromatography on SiO₂ using EtOAc (40-70%) in heptanes as the eluent to give the title compound.

Yield: 5.21 g, 18.1 mmol, 65%. MS (ESI, pos): 310.2 [M+Na]+, 597.4 [2M+Na]⁺

### Example 1 k)

### Synthesis of 3-(Benzyloxy)-1-methyl-2-oxo-1,2-dihydropyridine-4-carboxylic acid

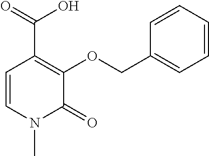 [SMILES: Cn1ccc(C(=O)O)c(OCc2ccccc2)c1=O]

{ethyl 3-(benzyloxy)-1-methyl-2-oxo-1,2-dihydropyridine-4-carboxylate} (27.90 g, 97.1 mmol) was dissolved in 250 mL MeOH and 60 mL NaOH (5M, aq) was added. The reaction mixture was stirred for 2 hours at room temperature before the reaction mixture was concentrated to approximately ⅓ in vacuo. The residue was diluted with 150 mL water and acidified to pH 2 using hydrogen chloride (HCl) (5M, aq). The precipitate was filtered and dried in vacuo, giving the title compound as a colorless solid. Yield: 22.52 g, 86.9 mmol, 89%.

### Example 1 l)

### Synthesis of 3-(Benzyloxy)-1-methyl-4-(2-thioxothiazolidine-3-carbonyl)pyridine-2 (1H)-one (AGC0021)

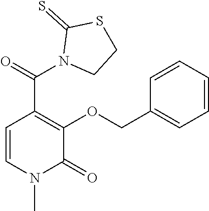 [SMILES: Cn1ccc(C(=O)N2CCSC2=S)c(OCc2ccccc2)c1=O]

{3-(benzyloxy)-1-methyl-2-oxo-1,2-dihydropyridine-4-carboxylic acid} (3.84 g, 14.8 mmol), 4-dimethylaminopyridine (DMAP) (196 mg, 1.6 mmol) and 2-thiazoline-2-thiol (1.94 g, 16.3 mmol) was dissolved in 50 mL DCM. N,N′-Dicyclohexylcarbodiimide (DCC) (3.36 g, 16.3 mmol) was added. The reaction mixture was stirred over night. The reaction was filtered, the solids washed with DCM and the filtrate was reduced in vacuo. The resulting yellow solid was recrystallized from isopropanol/DCM, giving AGC0021. Yield: 4.65 g, 12.9 mmol, 87%. MS(ESI, pos): 383 [M+Na]+, 743 [2M+Na]+

### Example 1 m)

### Synthesis of AGC0023

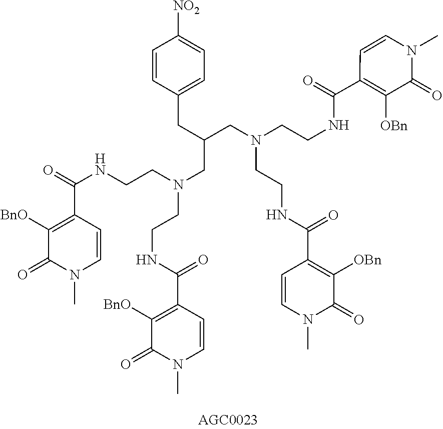 [SMILES: Cn1ccc(C(=O)NCCN(CCNC(=O)c2ccn(C)c(=O)c2OCc2ccccc2)CC(Cc2ccc([N+](=O)[O-])cc2)CN(CCNC(=O)c2ccn(C)c(=O)c2OCc2ccccc2)CCNC(=O)c2ccn(C)c(=O)c2OCc2ccccc2)c(OCc2ccccc2)c1=O]

AGC0020 (8.98 g; 23.5 mmol) was dissolved in CH₂Cl₂ (600 mL). AGC0021 (37.43 g; 103.8 mmol) was added. The reaction was stirred for 20 hours at room temperature. The reaction mixture was concentrated under reduced pressure.

DFC on SiO₂ using a gradient of methanol in a 1:1 mixture of EtOAc and CH₂Cl₂ yielded AGC0023 as a solid foam.

Average yield: 26.95 g, 20.0 mmol, 85%.

### Example 1 n)

### Synthesis of AGC0024

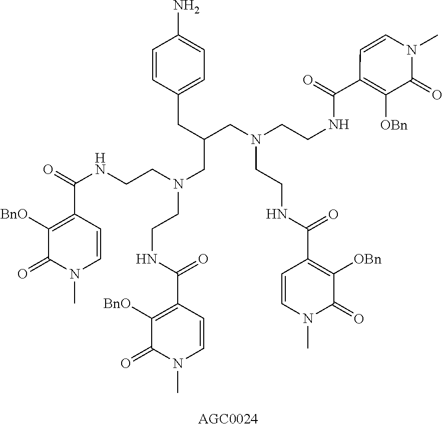 [SMILES: Cn1ccc(C(=O)NCCN(CCNC(=O)c2ccn(C)c(=O)c2OCc2ccccc2)CC(Cc2ccc(N)cc2)CN(CCNC(=O)c2ccn(C)c(=O)c2OCc2ccccc2)CCNC(=O)c2ccn(C)c(=O)c2OCc2ccccc2)c(OCc2ccccc2)c1=O]

AGC0023 (26.95 g; 20.0 mmol) was dissolved in ethanol (EtOH) (675 mL). Iron (20.76 g; 0.37 mol) and NH₄Cl (26.99 g; 0.50 mol) were added, followed by water (67 mL). The reaction mixture was stirred at 70° C. for two hours. More iron (6.75 g; 121 mmol) was added, and the reaction mixture was stirred for one hour at 74° C. More iron (6.76 g; 121 mmol) was added, and the reaction mixture was stirred for one hour at 74° C. The reaction mixture was cooled before the reaction mixture was reduced under reduced pressure.

DFC on SiO₂ using a gradient of methanol in CH₂Cl₂ yielded AGC0024 as a solid foam. Yield 18.64 g, 14.2 mmol, 71%.

### Example 1 o)

### Synthesis of AGC0025

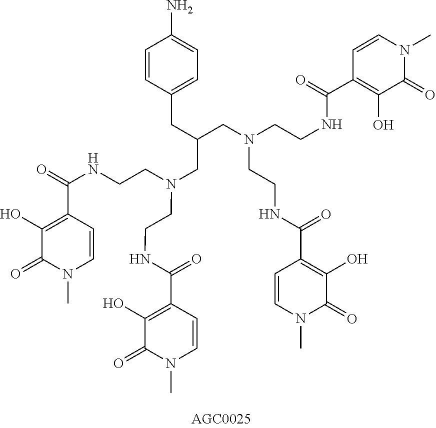 [SMILES: Cn1ccc(C(=O)NCCN(CCNC(=O)c2ccn(C)c(=O)c2O)CC(Cc2ccc(N)cc2)CN(CCNC(=O)c2ccn(C)c(=O)c2O)CCNC(=O)c2ccn(C)c(=O)c2O)c(O)c1=O]

AGC0024 (18.64 g; 14.2 mmol) was dissolved in CH₂Cl₂ (750 mL) and cooled to 0° C. BBr₃ (50 g; 0.20 mol) was added and the reaction mixture was stirred for 75 minutes. The reaction was quenched by careful addition of methanol (MeOH) (130 mL) while stirring at 0° C. The volatiles were removed under reduced pressure. HCl (1.25M in EtOH, 320 mL) was added to the residue. The flask was then spun using a rotary evaporator at atmospheric pressure and ambient temperature for 15 minutes before the volatiles were removed under reduced pressure.

DFC on non-endcapped C₁₈ silica using a gradient of acetonitrile (ACN) in water yielded AGC0025 as a slightly orange glassy solid.

Yield 13.27 g, 13.9 mmol, 98%.

### Example 1 p)

### Synthesis of AGC0019

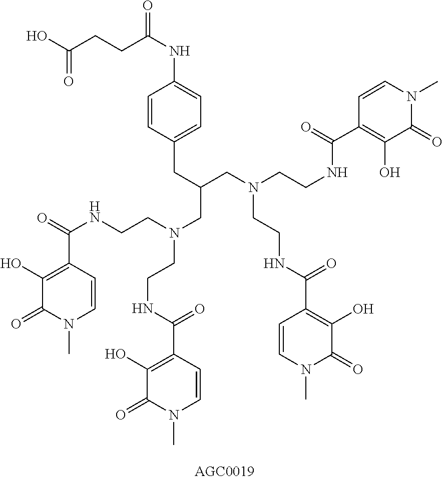 [SMILES: Cn1ccc(C(=O)NCCN(CCNC(=O)c2ccn(C)c(=O)c2O)CC(Cc2ccc(NC(=O)CCC(=O)O)cc2)CN(CCNC(=O)c2ccn(C)c(=O)c2O)CCNC(=O)c2ccn(C)c(=O)c2O)c(O)c1=O]

AGC0025 (10.63 g; 11.1 mmol) was dissolved in ACN (204 mL) and water (61 mL) at room temperature. Succinic anhydride (2.17 g; 21.7 mmol) was added and the reaction mixture was stirred for two hours. The reaction mixture was reduced under reduced pressure. DFC on non-endcapped C₁₈ silica using a gradient of ACN in water yielded a greenish glassy solid.

The solid was dissolved in MeOH (62 mL) and water (10.6 mL) at 40° C. The solution was added drop wise to EtOAc (750 mL) under sonication. The precipitate was filtered, washed with EtOAc and dried under reduced pressure, giving AGC0019 as an off-white solid with a greenish tinge.

Yield: 9.20 g, 8.7 mmol, 78%. H-NMR (400 MHz, DMSO-d₆), ¹³C-NMR (100 MHz, DMSO-d₆).

### Example 2

### Isolation of Pure Thorium-227

Thorium-227 is isolated from an actinium-227 generator. Actinium-227 was produced through thermal neutron irradiation of Radium-226 followed by the decay of Radium-227 (t½=42.2 m) to Actinium-227. Thorium-227 was selectively retained from an Actinium-227 decay mixture in 8 M HNO₃ solution by anion exchange chromatography. A column of 2 mm internal diameter, length 30 mm, containing 70 mg of AG®1-X8 resin (200-400 mesh, nitrate form) was used. After Actinium-227, Radium-223 and daughters had eluted from the column, Thorium-227 was extracted from the column with 12 M HCl. The eluate containing Thorium-227 was evaporated to dryness and the residue resuspended in 0.01 M HCl prior to labelling step.

### Example 3

### Cytotoxicity of ²²⁷Th-AGC1118 Against Ramos

### Example 3 a)

### Generation of the Anti-CD22 Monoclonal Antibody (AGC1100)

The sequence of the monoclonal antibody (mAb) hLL2, also called epratuzumab, here denoted AGC1100, was constructed as described in Leung, Goldenberg, Dion, Pellegrini, Shevitz, Shih, and Hansen: Molecular Immunology 32: 1413-27, 1995.

The mAb used in the current examples was produced by Immunomedics Inc, New Jersey, USA. Production of this mAb could for example be done in Chinese hamster ovarian suspension (CHO-S) cells, transfected with a plasmid encoding the genes encoding the light and the heavy chain. First stable clones would be selected for using standard procedures. Following approximately 14 days in a single-use bioreactor, the monoclonal antibody may be harvested after filtration of the supernatant. AGC1100 would be further purified by protein A affinity chromatography (MabSelect SuRe, Atoll, Weingarten/Germany), followed by an ion exchange step. A third purification step based on electrostatic and hydrophobicity could be used to remove aggregates and potentially remaining impurities. The identity of AGC1100 would be confirmed by isoelectric focusing, SDS-PAGE analysis, N-terminal sequencing and LC/MS analysis. Sample purity would be further analyzed by size-exclusion chromatography (SEC).

### Example 3 b) Coupling of mAb AGC1100 (Epratuzumab) with the Chelator AGC0019 (Compound of Formula (VIII)) to Give Conjugate AGC1118

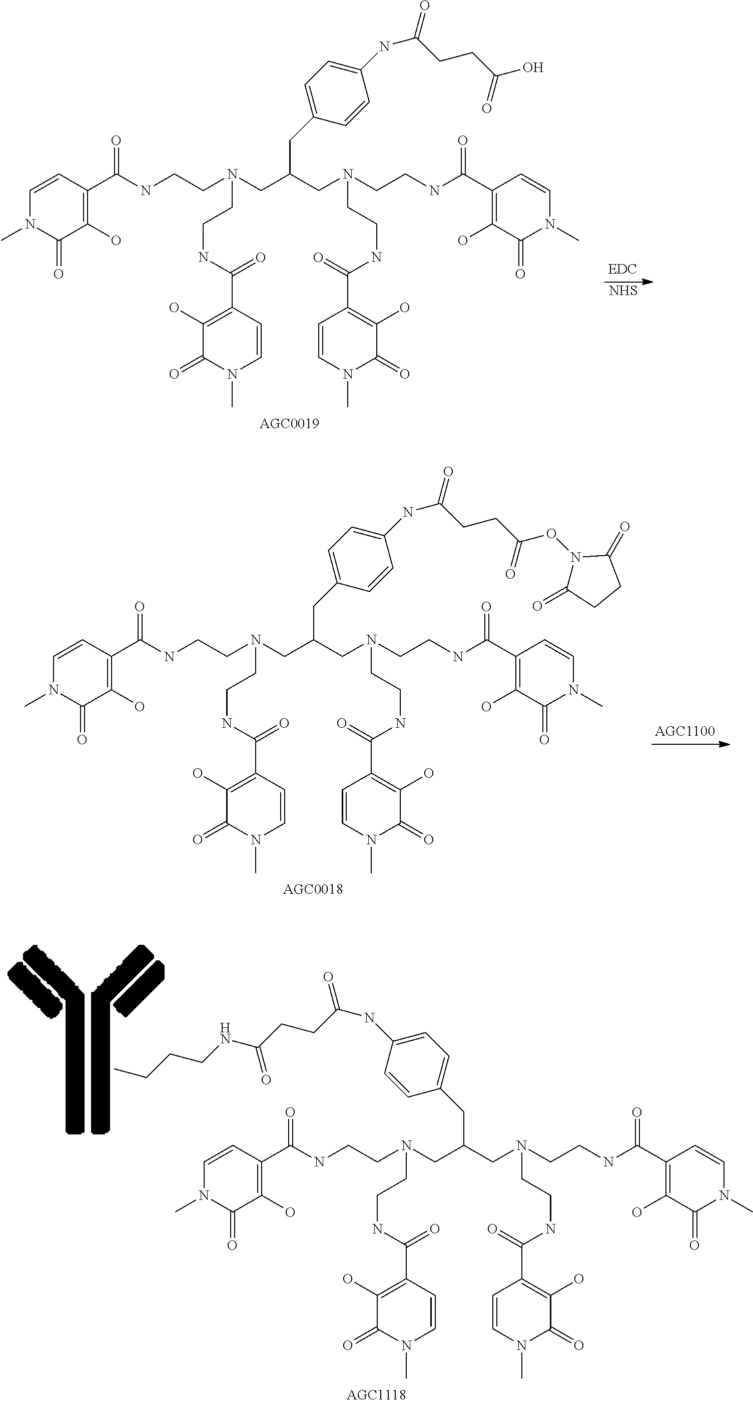

Prior to conjugation, phosphate buffer pH 7.5 was added to the antibody solution (AGC1100) to increase the buffering capacity of the solution. The amount of AGC1100 (mAb) in the vessel was determined.

The chelator AGC0019 was dissolved in 1:1, DMA: 0.1 M MES buffer pH 5.4. NHS and EDC were dissolved in 0.1 M MES buffer pH 5.4.

A 1/1/3 molar equivalent solution of chelator/N-hydroxysuccinimide (NHS)/1-ethyl-3-(3-dimethylaminopropyl)carbodiimide (EDC) was prepared to activate the chelator. For conjugation to the antibody a molar ratio of 7.5/7.5/22.5/1 (chelator/NHS/EDC/mAb) of the activated chelator was charged to mAb. After 20-40 minutes, the conjugation reaction was quenched with 12% v/v 0.3M Citric acid to adjust pH to 5.5.

The solution was then buffer exchanged into 30 mM Citrate, 70 mM NaCl, 2 mM EDTA, 0.5 mg/ml pABA, pH 5.5 (TFF Buffer) by Tangential Flow Filtration at constant volume. At the end of diafiltration the solution was discharged to a formulation container. The product was formulated with TFF buffer (30 mM Citrate, 70 mM NaCl, 2 mM EDTA, 0.5 mg/ml pABA, pH 5.5) and 7% w/v polysorbate 80 to obtain 2.6 mg/mL AGC1118 in 30 mM citrate, 70 mM NaCl, 2 mM EDTA, 0.5 mg/mL pABA 0.1% w/v PS80, pH 5.5. Finally, the solution was filtered through a 0.2 μm filter into sterile bottles prior to storage.

### Example 3 c)

### Preparation of a Dose on ²²⁷Th-AGC1118 Injection

A vial of 20 MBq thorium-227 chloride film was dissolved in 2 ml 8M HNO₃ solution and left for 15 minutes before withdrawing the solution for application to an anion exchange column for removal of radium-223 that had grown in over time. The column was washed with 3 ml 8M HNO₃ and 1 ml water prior to elution of thorium-227 with 3 ml 3M HCl. The eluted activity of thorium-227 was measured and a dose of 10 MBq transferred to an empty 10 ml glass vial. The acid was then evaporated using a vacuum pump and having the vial in a heating block (set to 120° C.) for 30-60 minutes. After reaching room temperature, 6 ml AGC1118 conjugate 2.5 mg/ml was added for radiolabelling. The vial was gently mixed and left for 15 minutes at room temperature. The solution was then sterile filtered into a sterile vial and sample withdrawn for iTLC analysis to determine RCP before use.

### Example 3 d)

### Cytotoxicity of ²²⁷Th-AGC1118 Against Ramos with Different Total Activity and Specific Activity

In this study doses of ²²⁷Th-AGC1118 were tested by varying total activity and specific activity with 4 hours incubation time. This study was run in a 96 well plate format at specific activity at 10/50 kBq/μg and total activity at 5, 10, 20 and 40 kBq/ml.

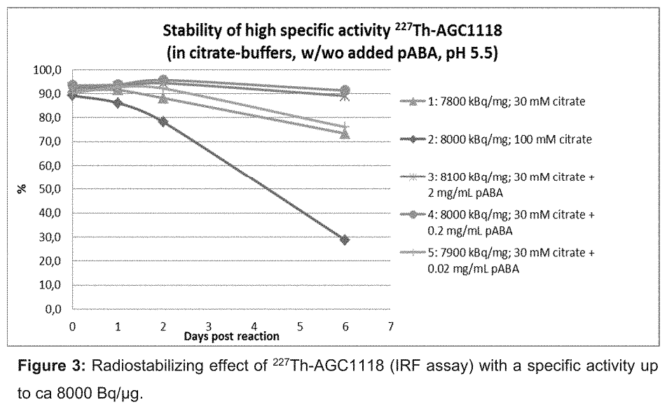

Ramos cells were cultured in RPM11640-medium with 10% FBS and 1 Pencillin/Streptomycin (Passage 22). Cells were transferred to a centrifuge tube and centrifuged at 300 G for 5 minutes and suspend in 5 mL medium before counting on a Z2 Coulter Counter. The cell suspension was diluted with medium to a cell concentration of 400.000 cells/ml and transferred to 48 wells (200 μl/well) in a 96 well plate (80.000 cells/well). CellTiter-Glo Luminescent Cell Viability Assay (Promega) was used for measuring cell viability. See FIG. 4.

### Example 4

### Cytotoxicity of ²²⁷Th-AGC0718 Against HL-60

### Example 4 a)

### Generation of the Anti-CD33 Monoclonal Antibody (AGC0700)

The sequence of the monoclonal antibody (mAb) HuM195/lintuzumab, here denoted as AGC0700, was retrieved from the literature as described in (1) and (2). Manufacturing of AGC0700 was conducted at the facilities of CobraBiologics (Södertälje, Sweden). Briefly, the amino acid sequences of heavy- and light-chains were back-translated into DNA sequence using Vector NTI® Software (Invitrogen/Life-Technologies Ltd., Paisley, United Kingdom). The codon for the C-terminal lysine (Lys) was omitted from the IgG1 heavy chain gene to facilitate precise determination of the conjugate to antibody ratio (CAR) as outlined in Example 2. The resulting DNA sequence was codon optimized for expression in mammalian cells and synthesized by GeneArt (GeneArt/Life-Technologies Ltd., Paisley, United Kingdom) and further cloned into an expression vector by CobraBiologics (Södertälje, Sweden). Chinese hamster ovarian suspension (CHO-S) cells were stably transfected with the plasmid encoding the V_{H}- and V_{L}-domains of AGC0700 and grown in presence of standard CD-CHO medium (Invitrogen/Life-Technologies Ltd., Paisley, United Kingdom), supplemented with puromycin (12.5 mg/l; Sigma Aldrich). Stable clones, expressing AGC0700, were selected via limiting dilution over 25 generations. Clone stability was assessed by measuring protein titers from supernatants. A cell bank of the most stable clone was established and cryo-preserved.

Expression of the mAb was carried out at 37° C. for approximately 14 days in a single-use bioreactor at a 250 L scale. The monoclonal antibody was harvested after filtration of the supernatant. AC0700 was further purified via a protein A affinity column (MabSelect SuRe, Atoll, Weingarten/Germany), followed by one anion (QFF-Sepharose; GE Healthcare)—and a cation (PorosXS; Invitrogen/Life-Technologies Ltd.)—exchange chromatography to increase purity and final yield. The identity of AGC0700 was confirmed by isoelectric focusing and SDS-PAGE analysis. Activity of purified AGC0700 was analyzed in a binding ELISA to immobilized CD33-Fc target (Novoprotein). Sample purity was analyzed by size-exclusion chromatography (SEC).

### REFERENCES

(1) Scheinberg D A. “Therapeutic uses of the hypervariable region of monoclonal antibody M195 and constructs thereof. U.S. Pat. No. 6,007,814 (1999 Dec. 28).
(2) Co M S et al; J Immunol. 1992 Feb. 15; 148(4):1149-54. Chimeric and humanized antibodies with specificity for the CD33 antigen.

### Example 4 b)

### Coupling of mAb AGC0700 (Lintuzumab) with the Chelator AGC0019 (Compound of Formula (VIII)) to Give Conjugate AGC0718

Conjugations were performed as described in example 3 with minor exceptions.

Prior to conjugation, phosphate buffer pH 7.5 was added to the antibody solution (AGC0700) to increase the buffering capacity of the solution. The amount of AGC0700 (mAb) in the vessel was determined.

The chelator AGC0019 was dissolved in 1:1, DMA: 0.1 M MES buffer pH 5.4. NHS and EDC were dissolved in 0.1 M MES buffer pH 5.4.

A 1/1/3 molar equivalent solution of chelator/NHS/EDC was prepared to activate the chelator. For conjugation to the antibody a molar ratio of 20/20/60/1 (chelator/NHS/EDC/mAb) of the activated chelator was charged to mAb. After 40-60 minutes, the conjugation reaction was quenched with 12% v/v 0.3M Citric acid to adjust pH to 5.5.

The solution was then buffer exchanged into 30 mM Citrate, 154 mM NaCl, 2 mM EDTA, 2 mg/ml pABA, pH 5.5 (TFF Buffer) by Tangential Flow Filtration at constant volume. At the end of diafiltration the solution was discharged to a formulation container. The product was formulated with TFF buffer (30 mM Citrate, 154 mM NaCl, 2 mM EDTA, 2 mg/ml pABA, pH 5.5) to obtain 2.5 mg/mL AGC0718 in 30 mM citrate, 154 mM NaCl, 2 mM EDTA, 2 mg/mL pABA, pH 5.5. Finally, the solution was filtered through a 0.2 μm filter into sterile bottles prior to storage.

### Example 4 c)

### Preparation of a Dose on ²²⁷Th-AGC0718 Injection

A vial of 20 MBq thorium-227 chloride film was dissolved in 2 ml 8M HNO3 solution and left for 15 minutes before withdrawing the solution for application to an anion exchange column for removal of radium-223 that had grown in over time. The column was washed with 3 ml 8M HNO3 and 1 ml water prior to elution of thorium-227 with 3 ml 3M HCl. The eluted activity of thorium-227 was measured and a dose of 10 MBq transferred to an empty 10 ml glass vial. The acid was then evaporated using a vacuum pump and having the vial in a heating block (set to 120° C.) for 30-60 minutes. After reaching room temperature, 6 ml AGC0718 conjugate 2.5 mg/ml was added for radiolabelling. The vial was gently mixed and left for 15 minutes at room temperature. The solution was then sterile filtered into a sterile vial and sample withdrawn for iTLC analysis to determine RCP before use.

### Example 4 d)

### Cytotoxicity of ²²⁷Th-AGC0718 Against HL-60 with Different Total Activity

To demonstrate cell toxicity of ²²⁷Th-AGC0718 after binding to CD33+-cells, in vitro cell toxicity assays were performed. For this purpose, the human myelogenic leukemic HL-60 cell line, as well as a CD33-negative B-cell line (Ramos), were exposed to ²²⁷Th-AGC0718. Total activities of 2 and 20 kBq/ml were tested at a specific activity of 44 kBq/μg. All experimental procedures are described in RD2013.093. Briefly, 50 000 human HL-60 cells/ml in IMDM-medium were prepared with 10% FBS and 1 Penicillin/Streptomycin and seeded at a density of 100.000 cells/well in a 24 well plate. Cells were incubated for 4 h at 37° C. with activities of 0 to 20 kBq/ml of ²²⁷Th-AGC0718. A respective ²²⁷Th-isotype control conjugate sample as well as ab unlabeled AGC0718 sample were prepared in parallel as respective controls. Cells were washed afterwards with fresh medium and seeded into a new 24-well culture plate.

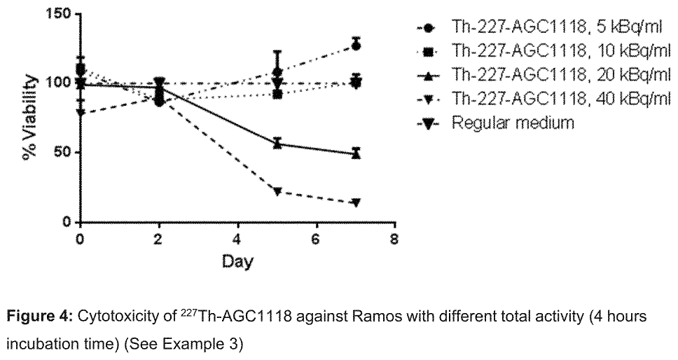

At different time points, cells were harvested and the viability was measured using the CellTiterGlo kit (Promega). The viability was expressed in % by setting the positive control (untreated cells) to 100%. See FIG. 5.

### Example 5

### Cytotoxicity of ²²⁷Th-AGC0118 Against SKOV-3

### Example 5 a)

### Generation of AGC0100 (Trastuzumab)

Trastuzumab monoclonal antibody (here denoted as AGC0100) was purchased from Roche and dissolved to a concentration of 10 mg/ml in PBS (Dulbecco BIOCHROM).

### Example 5 b)

### Coupling of mAb AGC0100 (Trastuzumab) with the Chelator AGC0019 (Compound of Formula (VIII)) to Give Conjugate AGC0118

Conjugations were performed as described in example 3 with minor modifications and. TFF purification of final conjugated mAb was replaced by gelfiltration column chromatography.

To trastuzumab in PBS was added 11% 1 M phosphate buffer pH 7.4. Chelator (AGC0019) NHS and EDC were dissolved in the same solutions as described in example 3 b). The molar ratio of chelator/NHS/EDC during activation was 1/1/3. A molar ratio of 8/8/25/1 corresponding to chelator/NHS/EDC/mAb and 30-40 min conjugation time, resulted in a CAR (chelator to antibody ratio) of 0.7-0.9 for conjugated AGC0118. The reaction was quenched by the addition of 12% v/v 0.3M citric acid to final pH of 5.5. Purification and buffer exchange of AGC0118 conjugates into 30 mM Citrate pH 5.5, 154 mM NaCl were performed by gelfiltration on a Superdex 200 (GE Healthcare) column connected to an ÄKTA system (GE Healthcare). The protein concentration at Abs 280 nm was measured before the product was formulated with buffer (to obtain 2.5 mg/mL AGC0118 in 30 mM citrate, 154 mM NaCl, 2 mM EDTA, 2 mg/mL pABA, pH 5.5). Finally, the solution was filtered through a 0.2 μm filter into sterile bottles prior to storage.

### Example 5 c)

### Preparation of a Dose on ²²⁷Th-AGC0118 Injection

Labelling was performed as previously described:

A vial of 20 MBq thorium-227 chloride film was dissolved in 2 ml 8M HNO3 solution and left for 15 minutes before withdrawing the solution for application to an anion exchange column for removal of radium-223 that had grown in over time. The column was washed with 3 ml 8M HNO3 and 1 ml water prior to elution of thorium-227 with 3 ml 3M HCl. The eluted activity of thorium-227 was measured and a dose of 10 MBq transferred to an empty 10 ml glass vial. The acid was then evaporated using a vacuum pump and having the vial in a heating block (set to 120° C.) for 30-60 minutes. After reaching room temperature, 6 ml AGC0118 conjugate 2.5 mg/ml was added for radiolabelling. The vial was gently mixed and left for 15 minutes at room temperature. The solution was then sterile filtered into a sterile vial and sample withdrawn for iTLC analysis to determine RCP before use.

### Example 5 d)

### Cytotoxicity of ²²⁷Th-AGC0118 Against SKOV-3 with Different Total Activity

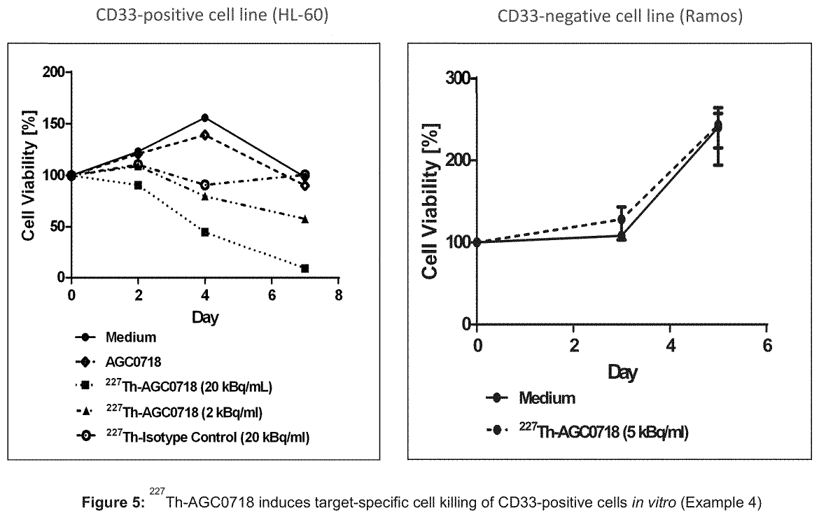

Cell cytotoxicity was tested to various doses of ²²⁷Th-AGC0118 by varying the total activity added to wells during 4 hours incubation time. SKOV-3 cells were seeded 10000 per well in a 96 well plate the day before experiment. A series of total activities 5, 10, 20 and 40 kBq/ml of chelated ²²⁷Th-AGC0118, at specific activity 20 kBq/μg, were added to the cells at day 1. Remaining non-bound ²²⁷Th-AGC0118 were removed by multi array pipette, followed by one additional wash with medium and subsequently fresh culture medium, after the end of incubation period. SKOV-3 cells were cultured in Mc-Coy medium with 10% FBS and 1% Penicillin/Streptomycin. Serum-free medium replaced the culture medium during the incubation with ²²⁷Th-AGC0118. At day four the CellTiter-Glo Luminescent Cell Viability Assay (Promega) was used for measuring cell viability. See FIG. 6.

### Example 6

### Cytotoxicity of ²²⁷Th-AGC2518 Against NCI-H716

### Example 6 a)

### Generation of the FGFR2 Monoclonal Antibody (BAY1179470; AGC2500)

The generation of the monoclonal antibody BAY 1179470, here further referred to AGC2500, is described in detail in WO2013076186A1. Briefly, the antibody was retrieved upon biopanning on FGFR2 antigen. The resulting human IgG1 antibody was expressed in CHO cells and purified using a protein A affinity column (MAb Select Sure), followed by size-exclusion chromatography to isolate monomeric fractions. The antibody was formulated into PBS, pH 7.4. Analytical SEC demonstrated homogeneity >99%.

### Example 6 b)

### Coupling of mAb AGC2500 with the Chelator AGC0019 (Compound of Formula (VIII)) to Give Conjugate AGC2518

The antibody-containing solution was adjusted to pH 7.5. The chelator AGC0019 was dissolved in 1:1, DMA: 0.1 M MES buffer pH 5.4. NHS and EDC were dissolved in 0.1 M MES buffer pH 5.4. A 1/1/3 molar equivalent solution of chelator/NHS/EDC was prepared to activate the chelator. For conjugation to the antibody a molar ratio of 10/10/30/1 (chelator/NHS/EDC/mAb) of the activated chelator was charged to mAb. After 30 minutes, the conjugation reaction was quenched with 12% v/v 0.3M Citric acid to adjust pH to 5.5. The reaction sample was further loaded on to a HiLoad 16/600 Superdex 200 (prep-grade) column to isolate monomeric fractions with 30 mM Citrate, 70 mM NaCl, pH 5.5 as mobile phase. At the end of the chromatography, the antibody conjugate AGC2518 was concentrated to 2.5 mg/ml in 30 mM Citrate, 70 mM NaCl, 2 mM EDTA and 0.5 mg/ml pABA. All procedures are described in RD.2014.092, Journal No. 211/149, 140619 AEF.

### Example 6 c)

### Preparation of a Dose on ²²⁷Th-AGC2518 Injection

A vial of 20 MBq thorium-227 chloride film was dissolved in 2 ml 8M HNO3 solution and left for 15 minutes before withdrawing the solution for application to an anion exchange column for removal of radium-223 that had grown in over time. The column was washed with 3 ml 8M HNO3 and 1 ml water prior to elution of thorium-227 with 3 ml 3M HCl. The eluted activity of thorium-227 was measured and a dose of 10 MBq transferred to an empty 10 ml glass vial. The acid was then evaporated using a vacuum pump and having the vial in a heating block (set to 120° C.) for 30-60 minutes. After reaching room temperature, 6 ml AGC2518 conjugate 2.5 mg/ml was added for radiolabelling. The vial was gently mixed and left for 15 minutes at room temperature. The solution was then sterile filtered into a sterile vial and sample withdrawn for iTLC analysis to determine RCP before use.

### Example 6 d)

### Cytotoxicity of ²²⁷Th-AGC2518 Against NCI-H716 Cells with Different Total Activities

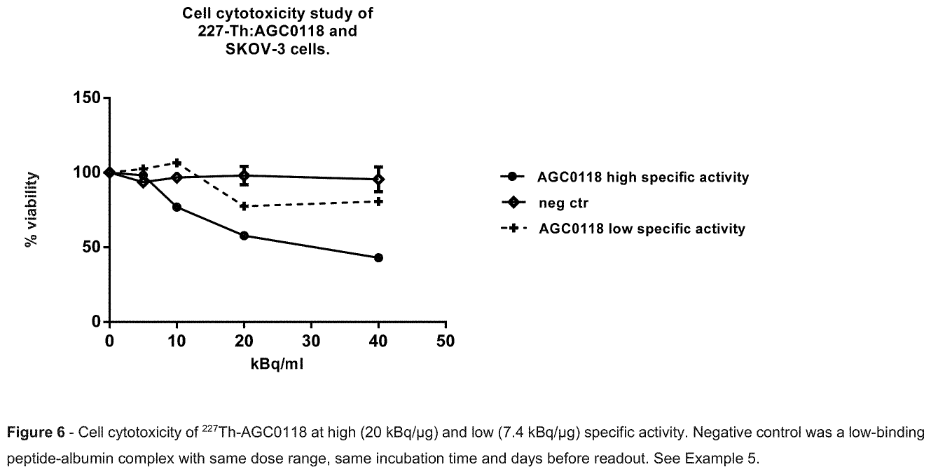

To demonstrate cell toxicity of ²²⁷Th-AGC2518 after binding to FGFR2+-cells, in vitro cell toxicity assays were performed. For this purpose, the human colorectal cancer cell line NCI-H716 was exposed to ²²⁷Th-AGC2518. Total activities of 2, 10, 20 and 40 kBq/ml were tested at a specific activity of 2 kBq/μg. An unrelated isotype control was prepared similar in parallel. All experimental procedures are described in RD2014.138. Briefly, 400 000 human NCI-H716 cells/ml in RPMI 1640-medium were prepared with 10% FBS and 1% Penicillin/Streptomycin and seeded at a density of 80.000 cells/well in a 96 well plate. Cells were incubated for 30 min at 37° C. with activities of 0 to 40 kBq/ml of ²²⁷Th-AGC2518 and a respective ²²⁷Th-isotype control conjugate sample. Cells were washed afterwards with fresh medium and seeded into a new 96-well culture plate. After 5 and 7 days, cells were harvested and the viability was measured using the CellTiterGlo kit (Promega). The viability was expressed in % by setting the positive control (untreated cells) to 100%. See FIG. 7.

### Example 7

### Cytotoxicity of ²²⁷Th-AGC2418 Against HT29 Cells

### Example 7 a)

### Generation of the Mesothelin Monoclonal Antibody (BAY 86-1903; AGC2400)

The generation of the monoclonal antibody BAY 86-1903, here further referred to AGC2400, is described in detail in WO2009068204. Briefly, the antibody was retrieved upon biopanning on Mesothelin antigen. The resulting human IgG1 antibody was expressed in CHO cells and purified using a protein A affinity column (MAb Select Sure), followed by aggregate removal using a HIC column (Toyopearl Butyl 600M). The antibody was formulated into PBS, pH 7.5.

### Example 7 b)

### Coupling of mAb AGC2400 with the Chelator AGC0019 (Compound of Formula (VIII)) to Give Conjugate AGC2418

The antibody-containing solution was adjusted to pH 7.5. The chelator AGC0019 was dissolved in 1:1, DMA: 0.1 M MES buffer pH 5.4. NHS and EDC were dissolved in 0.1 M MES buffer pH 5.4. A 1/1/3 molar equivalent solution of chelator/NHS/EDC was prepared to activate the chelator. For conjugation to the antibody a molar ratio of 16.5/16.5/49.5/1 (chelator/NHS/EDC/mAb) of the activated chelator was charged to mAb. After 30 minutes, the conjugation reaction was quenched with 12% v/v 0.3M Citric acid to adjust pH to 5.5. The reaction sample was further loaded on to a HiLoad 16/600 Superdex 200 (prep-grade) column to isolate monomeric fractions with 30 mM Citrate, 70 mM NaCl, pH 5.5 as mobile phase. At the end of the chromatography, the antibody conjugate AGC2418 was concentrated to 2.5 mg/ml in 30 mM Citrate, 70 mM NaCl, 2 mM EDTA and 0.5 mg/ml pABA. All procedures are described in RD.2014.111, Journal No. 211/160, 140814 AEF.

### Example 7 c)

### Preparation of a Dose on ²²⁷Th-AGC2418

A vial of 20 MBq thorium-227 chloride film was dissolved in 2 ml 8M HNO3 solution and left for 15 minutes before withdrawing the solution for application to an anion exchange column for removal of radium-223 that had grown in over time. The column was washed with 3 ml 8M HNO3 and 1 ml water prior to elution of thorium-227 with 3 ml 3M HCl. The eluted activity of thorium-227 was measured and a dose of 10 MBq transferred to an empty 10 ml glass vial. The acid was then evaporated using a vacuum pump and having the vial in a heating block (set to 120° C.) for 30-60 minutes. After reaching room temperature, 6 ml AGC2418 conjugate 2.5 mg/ml was added for radiolabelling. The vial was gently mixed and left for 15 minutes at room temperature. The solution was then sterile filtered into a sterile vial and sample withdrawn for iTLC analysis to determine RCP before use.

### Example 7 d)

### Cytotoxicity of ²²⁷Th-AGC2418 Against HT29 Cells, Overexpressing Mesothelin Antigen, with Different Total Activities

To demonstrate cell toxicity of ²²⁷Th-AGC2418 after binding to Mesothelin+-cells, in vitro cell toxicity assays were performed. For this purpose, the human colorectal cancer cell line HT29, transfected with the Mesothelin antigen, was exposed to ²²⁷Th-AGC2418. Total activities were titrated over 12 points in a threefold dilution, starting at 5 kBq/ml at a specific activity of 10 kBq/μg. An unrelated isotype control was prepared similar in parallel. All experimental procedures are described in RD2014.154. Briefly, 200 000 human HT29 cells, transfected with Mesothelin antigen, cells/ml in RPMI 1640-medium were prepared with 10% FBS, 1% Penicillin/Streptomycin, 1% NaHCO₃, 600 μg/ml Hygromycin B and seeded at a density of 40.000 cells/well in a 96 well plate. Cells were incubated for 6 days at 37° C. with activities of 0 to 40 kBq/ml of ²²⁷Th-AGC2418 and a respective ²²⁷Th-isotype control conjugate sample. At Day 6, cells were harvested and the viability was measured using the CellTiterGlo kit (Promega). The viability was expressed in % by setting the positive control (untreated cells) to 100%.

### Example 8

### Comparison of Stability of Amide and Isothiocyanate-Linked Conjugates

AGC1118 and the corresponding conjugate having an isothiocyanate coupling moiety (AGC1115) were stored in aqueous solution at 40° C. for 11 days. Samples were taken periodically.

40° C. samples normalized to each 4° C. sampling point

| | AGC1118 | AGC1115 |
|---|---|---|
| | CAR (% norm) | CAR (% norm) |
| Day 0 | 100 | 100 |
| Day 5 | 105 | 92 |
| Day 11 | 103 | 88 |

It can be seen from the above table that no measurable decrease in conjugate concentration was seen for the amide-coupled conjugate. In contrast, the isothiocyanate conjugate decreased by 8% after 5 days and by 12% after 11 days.

### Example 9

### Example 9 a)

### Generation of the PSMA Monoclonal Antibody (AGC1000)

The PSMA monoclonal antibody, hereinafter referred to as AGC1000, was purchased from Progenics, USA.

### Example 9 b)

### Coupling of mAb AGC1000 with the Chelator AGC0019 (Compound of Formula (VIII)) to Give Conjugate AGC1018

The antibody-containing solution was adjusted to pH 7.5. The chelator AGC0019 was dissolved in 1:1, DMA: 0.1M MES buffer pH 5.5. NHS and EDC were dissolved in 0.1M MES buffer pH 5.5. A 1/1/2 molar equivalent solution of chelator/NHS/EDC was prepared to activate the chelator. For conjugation to the antibody a molar ratio of 20/20/40/1 (chelator/NHS/EDC/mAb) of the activated chelator was charged to mAb in 4 portions with 10 minutes between each portion. After 50 minutes, the conjugation reaction was quenched with 12% v/v 1M TRIS pH 7.3. The conjugate was purified and buffer exchanged by tangential flow filtration (TFF). The formulation buffer was 30 mM Citrate, 70 mM NaCl, 2 mM EDTA, 0.5 mg/ml pABA, pH 5.5. At the end of diafiltration the solution was discharged to a bulk container and the concentration was adjusted to 2.7 mg/ml. Finally, the bulk solution was filtered through a 0.2 μm sterile filter and transferred to sterile vials for storage at −20° C.

### Example 9 c)

### Preparation of a Dose of ²²⁷Th-AGC1018 Injection

A vial of approx. 50 MBq Th-227 chloride film was dissolved in 2 ml 8M HNO₃ solution and left for 15 minutes before withdrawing the solution for application to an anion exchange column for removal of radium-223 that had grown in over time. The column was washed with 3 ml 8M HNO₃ and 1 ml water prior to elution of Th-227 with 3 ml 3M HCl. The HCl eluate was evaporated using a vacuum pump and a heating block set to 100° C. for 60-90 minutes. The activity of the dried Th-227 was measured in a dose calibrator. The dry Th-227 was dissolved in 0.05M HCl to give a concentration of 0.5 MBq/μl. For radiolabelling, the conjugate AGC1018 was diluted in formulation buffer in order to achieve 25 μg mAb in 200 μl. To the AGC1018 solution, 1 MBq Th-227 was mixed and the exact Th-227 activity measured on a Germanium detector. Chelation was allowed for 30-60 minutes at room temperature before sterile filtration into a sterile vial. A sample was withdrawn for iTLC analysis to determine RCP before use.

### Example 9 d)

### Cytotoxicity of ²²⁷Th-AGC1018 Against PSMA Expressing LNCaP Cells

To demonstrate cell toxicity of ²²⁷Th-AGC1018 after binding to PSMA positive cells, in vitro cell toxicity assays were performed. For this purpose, the human prostate cancer cell line LNCaP was exposed to ²²⁷Th-AGC1018. Total activities were titrated over 12 points in a threefold dilution, starting at 20 kBq/ml at a specific activity of 40 kBq/μg. An unrelated isotype control was prepared in parallel. All experimental procedures are described in archive RD.2015.101. Briefly, human LNCaP cells were cultured in RPMI 1640-medium supplemented with 10% FBS and 1% Penicillin/Streptomycin. Cells were seeded at a density of 2500 cells/well in a 96 well plate. 24 hours after seeding (Day 1), the cells were exposed to ²²⁷Th-AGC1018 and ²²⁷Th-isotype control at total activities ranging from 0 to 20 kBq/ml for 5 days at 37° C. At Day 6, cells were harvested and the viability was measured using the CellTiterGlo kit (Promega). The viability was expressed in % by setting the positive control (untreated cells) to 100%.

1. A method for the formation of a tissue-targeting thorium complex, said method comprising:
a) forming an octadentate chelator comprising four hydroxypyridinone (HOPO) moieties, substituted in the N-position with a C₁-C₃ alkyl group, and a coupling moiety terminating in a carboxylic acid group;
b) coupling said octadentate chelator to at least one tissue-targeting peptide or protein comprising at least one amine moiety by means of at least one amide-coupling reagent whereby to generate a tissue-targeting chelator; and
c) contacting said tissue-targeting chelator with an aqueous solution comprising an ion of at least one alpha-emitting thorium isotope.

2. The method of claim 1, wherein step b) is conducted in aqueous solution.

3. The method of claim 1, wherein said amide-coupling reagent is functional in aqueous solution.

4. The method of claim 1, wherein said amide-coupling reagent is a carbodiimide coupling reagent.

5. The method of claim 1, wherein step b) is conducted in aqueous solution at pH between 4 and 9.

6. The method of claim 1, wherein step b) is conducted between 15 and 50° C. for 5 to 120 minutes.

7. The method of claim 1, wherein step c) is conducted between 15 and 50° C. for 1 to 60 minutes.

8. The method of claim 1, wherein said octadentate chelator comprises four 3,2-HOPO moieties.

9. The method of claim 1, wherein said octadentate chelator is selected from formulae (VIb) and (VII): 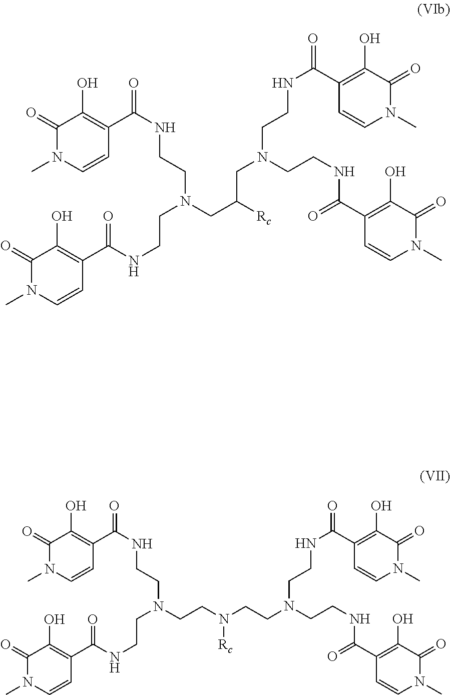
wherein R_{C} is a linker moiety terminating in a carboxylic acid moiety.

10. The method of claim 1, wherein said tissue-targeting moiety is a monoclonal or polyclonal antibody, an antibody fragment, or a construct of such antibodies or fragments, or a combination thereof.

11. The method of claim 1, wherein said tissue-targeting moiety has binding affinity for the CD22 receptor, FGFR2, Mesothelin, HER-2, PSMA, or CD33.

12. A tissue-targeting thorium complex formed or formable by the method of claim 1.

13. The tissue-targeting thorium complex of claim 12, comprising four 3,2-HOPO moieties.

14. The tissue-targeting thorium complex of claim 12, having binding affinity for the CD22 receptor, FGFR2, Mesothelin, HER-2, PSMA, or CD33.

15. The tissue-targeting thorium complex of claim 12, comprising the 4+ ion of an alpha-emitting thorium radionuclide.

16. The tissue-targeting thorium complex of claim 12, comprising an octadentate chelator of formula (VIb) or (VII): 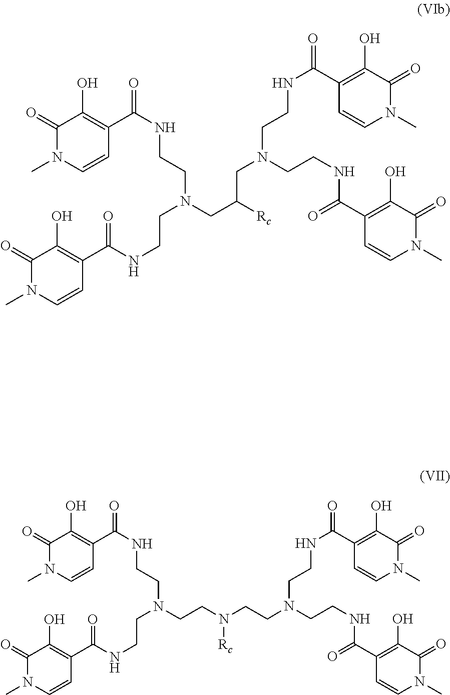
wherein R_{C} is a coupling moiety joined by an amide group to a tissue targeting moiety.

17. The tissue-targeting thorium complex of claim 12, comprising a tissue targeting moiety selected from the group consisting of a monoclonal or polyclonal antibody, an antibody fragment, and a construct of such antibodies or fragments, or a combination thereof.

18. The tissue-targeting thorium complex of claim 12, comprising a tissue targeting moiety comprising at least one peptide chain having at least 90% sequence similarity with at least one of the following sequences:
Light Chain:
(SEQ ID NO: 1)
DIQLTQSPSSLAVSAGENVTMSCKSSQSVLYSANHKNYLAWYQQKPGQSP
KLLIYWASTRESGVPDRFTGSGSGTDFTLTISRVQVEDLAIYYCHQYLSS
WTFGGGTKLEIKR
(SEQ ID NO: 2)
DIQLTQSPSSLASAAVEDRTMSCKSSQSVLYSANHKNYLAWYQQKPGQKA
KLLIYWASTRESGVPSRFSGSGSGTDFTFTISSLQPEDIATYYCHQYLSS
WTFGGGTKLEIKR
HeavyChain:
(SEQ ID NO: 3)
QVQLQESGAELSKPGASVKMSCKASGYTFTSYWLHWIKQRPGQGLEWIGY
INPRNDYTEYNQNFKDKATLTADKSSSTAYMQLSSLTSEDSAVYYCARRD
ITTFYWGQGTTLTVSS
(SEQ ID NO: 4)
QVQLQQSGAEVKKPGSSVKVSCKASGYTFTSYWLHWVRQAPGQGLEWIGY
INPRNDYTEYNQNFKDKATITADESTNTAYMELSSLRSEDTAFYFCARRD
ITTFYWGQGTTVTVSS
(SEQ ID NO: 5)
QVQLVQSGAEVKKPGSSVKVSCKASGYTFTSYWLHWVRQAPGQGLEWIGY
INPRNDYTEYNQNFKDKATITADESTNTAYMELSSLRSEDTAFYFCARRD
ITTFYWGQGTTVTVSS.

19. A pharmaceutical formulation comprising at least one tissue-targeting thorium complex of claim 12.

20. The pharmaceutical formulation of claim 19, further comprising citrate buffer.

21. The pharmaceutical formulation of claim 19, further comprising p-aminobutyric acid (PABA).

22-23. (canceled)

24. A method of treatment of a disease in a human or non-human animal comprising administering at least one tissue-targeting thorium complex of claim 12.

25. The method of claim 24, wherein the disease is hyperplastic or neoplastic disease.

26. (canceled)

27. A kit, comprising:
i) an octadentate chelator comprising four hydroxypyridinone (HOPO) moieties, substituted in the N-position with a C₁-C₃ alkyl group, and coupling moiety terminating in a carboxylic acid group;
ii) at least one tissue-targeting peptide or protein comprising at least one amine moiety; and
iii) at least one amide-coupling reagent.

28. The method of claim 4, wherein the carbodiimide coupling reagent is selected from the group consisting of 1-ethyl-3-(3-dimethylaminopropyl)carbodiimid (EDC), N,N′-diisopropylcarbodiimid (DIC), and N,N′-dicyclohexylcarbodiimid (DCC).

29. The method of claim 9, wherein R_{C} is [—CH₂-Ph-N(H)—C(═O)—CH₂—CH₂—C(═O)OH], [—CH₂—CH₂—N(H)—C(═O)—(CH₂—CH₂—O)₁₋₃—CH₂—CH₂—C(═O)OH], or [—(CH₂)₁₋₃-Ph-N(H)—C(═O)—(CH₂)₁₋₅—C(═O)OH], wherein Ph is a phenylene group.

30. The method of claim 29, wherein the phenylene group is a para-phenylene group.

31. The method of claim 10, wherein the antibody fragment is Fab, F(ab′)₂, Fab′, or scFv.

32. The tissue-targeting thorium complex of claim 15, wherein the alpha-emitting thorium radionuclide is ²²⁷Th.

33. The tissue-targeting thorium complex of claim 16, wherein R_{C} is AGC0019.

34. The tissue-targeting thorium complex of claim 17, wherein the antibody fragment is Fab, F(ab′)₂, Fab′, or scFv.

35. The pharmaceutical composition of claim 21, further comprising EDTA or at least one polysorbate, or a combination thereof.

36. The method of claim 25, wherein the hyperplastic or neoplastic disease is carcinoma, sarcoma, myeloma, leukemia, lymphoma, or mixed type cancer.

37. The method of claim 25, wherein the hyperplastic or neoplastic disease is Non-Hodgkin's Lymphoma, B-cell neoplasms, breast cancer, endometrial cancer, gastric cancer, acute myeloid leukemia, prostate cancer, brain cancer, mesothelioma, ovarian cancer, lung cancer, or pancreatic cancer.

38. The kit of claim 27, further comprising an alpha-emitting thorium radionuclide.

39. The kit of claim 38, wherein the alpha-emitting thorium radionuclide is ²²⁷Th.

40. A method of treatment of a disease in a human or non-human animal comprising administering at least one pharmaceutical formulation of claim 19.

41. The method of claim 40, wherein the disease is hyperplastic or neoplastic disease.

42. The method of claim 41, wherein the hyperplastic or neoplastic disease is carcinoma, sarcoma, myeloma, leukemia, lymphoma, or mixed type cancer.

43. The method of claim 41, wherein the hyperplastic or neoplastic disease is Non-Hodgkin's Lymphoma, B-cell neoplasms, breast cancer, endometrial cancer, gastric cancer, acute myeloid leukemia, prostate cancer, brain cancer, mesothelioma, ovarian cancer, lung cancer, or pancreatic cancer.
